SEQUENCE LISTING (2 sequences)

SEQ ID NO: 1 - protein, 453 aa, Homo sapiens
MQGAQEASASEMLPLLLPLLWAGALAQERRFQLEGPESLTVQEGLCVLVPCRLPTTLPASYYGYGYWFLEGADVPVATNDPDEEVQEETRGRFHLLWDPRRKNCSLSIRDARRRDNAAYFFRLKSKWMKYGYTSSKLSVRVMALTHRPNISIPGTLESGHPSNLTCSVPWVCEQGTPPIFSWMSAAPTSLGPRTTQSSVLTITPRPQDHSTNLTCQVTFPGAGVTMERTIQLNVSYAPQKVAISIFQGNSAAFKILQNTSSLPVLEGQALRLLCDADGNPPAHLSWFQGFPALNATPISNTGVLELPQVGSAEEGDFTCRAQHPLGSLQISLSLFVHWKPEGRAGGVLGAVWGASITTLVFLCVCFIFRVKTRRKKAAQPVQNTDDVNPVMVSGSRGHQHQFQTGIVSDHPAEAGPISEDEQELHYAVLHFHKVQPQEPKVTDTEYSEIKIHK

SEQ ID NO: 2 - protein, 257 aa, Homo sapiens
MAPAMESPTLLCVALLFFAPDGVLAVPQKPKVSLNPPWNRIFKGENVTLTCNGNNFFEVSSTKWFHNGSLSEETNSSLNIVNAKFEDSGEYKCQHQQVNESEPVYLEVFSDWLLLQASAEVVMEGQPLFLRCHGWRNWDVYKVIYYKDGEALKYWYENHNISITNATVEDSGTYYCTGKVWQLDYESEPLNITVIKAPREKYWLQFFIPLLVVILFAVDTGLFISTQQQVTFLLKIKRTRKGFRLLNPHPKPNPKNN

(19) United States
(12) Patent Application Publication
(10) Pub. No.: US 2024/0091164 A1
(43) Pub. Date: Mar. 21, 2024

(54) SURFACE COATING PPSU NANOPARTICLES FOR THERAPEUTIC MODULATION OF MAST CELLS

(71) Applicant: Northwestern University, Evanston, IL (US)

(72) Inventors: Evan Alexander Scott, Evanston, IL (US); Bruce Scott Bochner, Evanston, IL (US); Clayton Hunter Rische, Evanston, IL (US)

(21) Appl. No.: 18/464,127

(22) Filed: Sep. 8, 2023

Related U.S. Application Data
(60) Provisional application No. 63/375,028, filed on Sep. 8, 2022.

(51) Int. Cl. A61K 9/51; A61P 37/08; C07K 16/28

(52) U.S. Cl. CPC A61K 9/5169; A61P 37/08; C07K 16/2803; C07K 16/2851

(57) ABSTRACT

The present disclosure provides methods of adsorbing proteins to the surface of a nanoparticle. Particularly, methods for adsorbing anti-Siglec-6 and anti-FcεRI antibodies to PPSU nanoparticles, are described. The disclosure further provides compositions comprising the nanoparticles are methods of their use to prevent mast cell secretion and anaphylaxis.

### CROSS-REFERENCE TO RELATED APPLICATIONS

This application claims priority to U.S. Provisional Application No. 63/375,028 filed on Sep. 8, 2022, the content of which is incorporated by reference in its entirety.

### STATEMENT REGARDING FEDERALLY SPONSORED RESEARCH

This invention was made with government support under 1R01EB030629-01A1 and 1R21AI59586-01 awarded by National Institutes of Health. The government has certain rights in the invention.

### REFERENCE TO A SEQUENCE LISTING

The contents of the electronic sequence listing (702581.02401.xml; Size: 3,353 bytes; and Date of Creation: Sep. 7, 2023) is herein incorporated by reference in its entirety.

### BACKGROUND OF THE INVENTION

Allergic diseases are a number of conditions caused by hypersensitivity of the immune system to typically harmless substances in the environment that affect more than 50 million Americans. Severe allergic reactions result in anaphylaxis and can be potentially life threating, sometimes requiring hospital visits in conjunction with emergency interventions such as the use of epinephrine autoinjectors. Diseases characterized as type I hypersensitive reactions include allergic rhinitis, food allergy, atopic dermatitis, and allergic asthma.

During type I hypersensitive reactions, B cells are stimulated (by CD4+Th2 cells) to produce IgE antibodies specific to an allergen. The IgE antibodies then bind to FcεRI receptors on the surface of tissue mast cells and blood basophils, resulting in their sensitization. Later exposure to the same allergen cross-links the bound IgE on sensitized cells, resulting in anaphylactic degranulation, also known as mast cell degranulation or mast cell secretion, which is the immediate and explosive release of pharmacologically active pre-formed mediators from storage granules and concurrent synthesis of inflammatory lipid mediators from arachidonic acid; some of these mediators include histamine, leukotriene (LTC₄ and LTD₄ and LTB₄), GM-CSF, TNFα, IL-3, IL-4, IL-5, IL-6, IL-10, and IL-13, chemokines including CCL2, CCL3, CCL5, and CXCL8, and growth factors such as SCF, FGF, VEGF, angiogenin, and prostaglandin, which act on proteins (e.g., G-protein coupled receptors) located on surrounding tissues. The principal effects of these products are vasodilation and smooth-muscle contraction.

Current anaphylaxis management approaches involve avoidance of triggers and the emergency use of epinephrine and other agents when reactions do occur. Most recently, tolerogenic and desensitization therapies have shown promise, but for the most part these treatments, if effective at all, rarely induce sustained allergen unresponsiveness and instead tend to improve the dose threshold at which an allergic subject reacts¹. None of the current therapies are capable of directly regulating mast cell behavior in a way that prevents mast cell reactivity to allergens. Therefore, effective and safe proactive pharmacologic approaches for selective, prophylactic targeting of mast cells to prevent their allergic activation are needed.

### SUMMARY OF THE INVENTION

In an aspect, provided herein is a method for adsorbing at least one protein onto the surface of a poly(propylene sulfone) (PPSU) homopolymer nanoparticle, the method comprising incubating the nanoparticle with the at least one protein in a buffer for between about 1 second and about 5 minutes at between about 20° C. and about 22° C. In an embodiment, the concentration of the at least one protein is higher than the concentration of the nanoparticle. In an embodiment, the concentration of the at least one protein is at least twice as high as the concentration of the nanoparticle. In an embodiment, the method further comprises washing the nanoparticle with the buffer between 1 and 3 times. In an embodiment, the method further comprises suspending the nanoparticle in the buffer. In an embodiment, the buffer comprises phosphate buffered saline (PBS). In an embodiment, the at least one protein is an antibody. In an embodiment, the at least one antibody comprises two antibodies. In an embodiment, the two antibodies comprise an anti-Siglec-6 antibody and an anti-FcεRI antibody. In an embodiment, the anti-Siglec-6 antibody is antibody clone 767329, and wherein the anti-FcεRI antibody is antibody clone AER-37 (CRA1). In an embodiment, the anti-Siglec-6 antibody is incubated at a density of between about 0.001 wt % and about 1000 wt % PPSU. In an embodiment, the anti-FcεRI antibody is incubated at a density of between about 0.001 wt % and about 1000 wt % PPSU. In an embodiment, after incubating the nanoparticle with the antibody, the method further comprises incubating the nanoparticle with bovine serum albumin (BSA). In an embodiment, the PPSU homopolymer comprises PPSU₂₀.

In another aspect, provided herein is a composition for reducing mast cell secretion, the composition comprising a PPSU homopolymer nanoparticle coated with at least one of an anti-Siglec-6 antibody and an anti-FcεRI antibody; and a pharmaceutically acceptable excipient, carrier or diluent. In an embodiment, the nanoparticle comprises the anti-Siglec-6 antibody and the anti-FcεRI antibody. In an embodiment, the nanoparticle comprises PPSU₂₀. In an embodiment, the anti-Siglec-6 antibody is antibody clone 767329, and wherein the anti-FcεRI antibody is antibody clone AER-37 (CRA1).

In another aspect, provided herein is a method for treating a type I hypersensitivity reaction in a subject in need thereof, the method comprising administering a therapeutically effective amount of the composition described herein. In an embodiment, the type I hypersensitivity reaction is anaphylaxis.

### BRIEF DESCRIPTION OF THE DRAWINGS

Non-limiting embodiments of the present invention will be described by way of example with reference to the accompanying figures, which are schematic and are not intended to be drawn to scale. In the figures, each identical or nearly identical component illustrated is typically represented by a single numeral. For purposes of clarity, not every component is labeled in every figure, nor is every component of each embodiment of the invention shown where illustration is not necessary to allow those of ordinary skill in the art to understand the invention.

This patent or application file contains at least one drawing executed in color. Copies of this patent or patent application publication with color drawing(s) will be provided by the Office upon request and payment of the necessary fee.

FIGS. 1A-1E illustrate the assembly of PPSU-antibody constructs, preparation of materials, and experimental design as described herein. FIG. 1A is a fluidics model of the appearance of a spherical PPSU nanoparticle described herein. FIG. 1B provides a schematic of mast cell activation when exposed to the PPSU nanoparticle. FIG. 1C illustrates preparation of loaded PPSU nanoparticles. FIG. 1D illustrates the design of humanized mast cell mouse anaphylaxis. FIG. 1E shows the isolation and culture of primary human skin mast cells (hsMCs).

FIGS. 2A-2E Protein-induced dipole rotation of interfacial propylene sulfone leads to heterogeneous PPSU surfaces that mimic protein surfaces. (FIG. 2A) Simultaneously recorded cryo-STEM and cryo-SEM images showing a typical PPSU hollow nanoparticle and its surface, respectively. (FIG. 2B) Schematic illustration of the dipole moment of amphiphilic propylene sulfone, the repeating unit of PPSU homopolymer (blue indicates hydrophilic and grey indicates hydrophobic for all the schemes in the work, unless stated otherwise). (FIG. 2C) Atomistic simulations demonstrating enhanced surface hydrophilicity as the aggregation of PPSU chains become disordered. PEG is included for comparison. (FIG. 2D) As seen in trypsin using glycine as the reference, the surfaces of water-soluble proteins are heterogeneous, with characteristic patch size distributions based on hydrophobicity. (FIG. 2E) Protein inducing the formation of locally heterogeneous PPSU surfaces that mimic protein surfaces. Hydrophobic interactions at the interface between this PPSU surface and the adsorbed protein can be weak enough not to outcompete the forces governing protein folding despite irreversible adsorption. Fast spreading of protein on a hydrophobic surface is included for comparison².

FIGS. 3A-3H Preparation and characterization of PPSU NPs. (FIG. 3A) Schematic illustration of the confined impingement jets mixer used for thorough hydration. (FIG. 3B) Typical process for the producing of PPSU NPs. (FIG. 3C) A representative TEM image of PPSU NPs in water (0.7 mg/mL). (FIG. 3D) A representative Cryo-TEM image of PPSU NPs in water (5 mg/mL). (FIG. 3E) Simultaneously recorded cryo-STEM (top) and cryo-SEM (below) images showing PPSU NPs (3 mg/mL in water) and their surfaces, respectively. (FIG. 3F) DLS size distribution of PPSU NPs by number (top) and intensity (below) in water (1 mg/mL). (FIG. 3G) Zeta potential of PPSU NPs measured in water and in phosphate buffered saline (PBS). Suspensions of PPSU NPs were prepared by mixing NPs in water with 10 vol. % of PBS (10×), followed by zeta potential measurements immediately. (FIG. 3H) Chemical degradation of PPSU NPs showing undetectable DMSO residual. The NPs were dialyzed in D₂O instead of in H₂O, then resuspended in D₂O with 40 wt. % of NaOD for ¹H NMR measurement. DMSO in D₂O with 40 wt. % of NaOD was included as a control. Scale bar=100 nm.

FIGS. 4A-4B Constructing hollow NPs in atomistic MD simulations. (FIG. 4A) The initial and the final structures of the 125-chain NP. (FIG. 4B) The initial and the final structures of the 600-chain NP. To illustrate the hollow structures, only around a half of the NPs is displayed in the snapshots. Blue: Oxygen and sulfur atoms; Grey: Carbon and hydrogen atoms. Water molecules are omitted for display.

FIG. 5 Atomistic simulations demonstrating enhanced surface hydrophilicity as the size of the PPSU aggregate increases. Coulombic (Coul) and Lennard-Jones (LJ) interactions represent the polar and nonpolar interaction between PPSU and water, respectively. The Coul/LJ ratios are used to quantify surface hydrophilicity. A higher ratio stands for favored polar interactions, thus supporting an elevated hydrophilicity. PEG is included for comparison. The final simulation snapshots are shown correspondingly.

FIGS. 6A-6B Incubation of PPSU NPs with proteins in PBS leading to protein-coated NPs in 5 min. (FIG. 6A) While the adsorption of negatively charged proteins in water by PPSU NPs is inhibited by electrostatic repulsion, PPSU NPs can irreversibly adsorb nonspecific proteins in PBS. Bovine serum albumin (BSA), hemoglobin (HBB), and lysozyme (LYZ) were used without dye labelling. pI=distinct isoelectric point. The inset shows photos for protein-coated NPs after removing the excess proteins (BSA and LYZ were labelled with FITC and RhB, respectively). (FIG. 6B) When incubated with 5 wt % of proteins in PBS, PPSU NPs adsorbed nearly 100% of the proteins.

FIGS. 7A-7E Atomistic explicit solvent simulations confirm the interfacial hydrophilic-hydrophobic transition within PPSU surface upon trypsin adsorption. (FIG. 7A) Simulation snapshot of equilibrated 600-chain NP adsorbed with all the 6 trypsin molecules (in cyan). The protein-NP interfaces are lubricated by water molecules, leading to the preserved structure of trypsin. The green cylinders refer to the water bridges connecting PPSU chains and trypsin via H-bonds. (FIG. 7B) The Lennard-Jones trypsin-NP interactions dominated over the trypsin-NP Coulombic interactions, supporting the hydrophobicity-driven feature of trypsin adsorption. (FIG. 7C) Orientations of the 6 trypsin molecules are non-specific upon adsorption. The adsorption sites and active sites on trypsin are colored in red and blue, respectively. (FIG. 7D) Percentage of sulfone groups at the trypsin-NP contact region revealing enhanced NP surface hydrophobicity after trypsin adsorption. (FIG. 7E) No significant (ns) differences in trypsin hydration were detected between the adsorbed trypsin and unbound trypsin. The numbers of trypsin-water H-bonds and of water neighbors of trypsin were calculated. Error bars represent standard deviations. Statistical significance is determined by Tukey-post hoc test.

FIG. 8 The adsorbed trypsin molecules preserving their 3D structure after adsorption. The RMSD (root-mean-square deviation) of each trypsin molecule during the simulation. A small RMSD of around 1.5 Å supports the retention of the trypsin structures.

FIG. 9 Analysis of trypsin surface domain compositions in contact with PPSU NP. No remarkable difference was observed between the 6 adsorbed trypsin molecules and the unbound trypsin (control), indicating random and non-specific binding.

FIG. 10 Surface restructuring of the NP at the PPSU-trypsin interface upon trypsin adsorption. A decrease in the amount of polar sulfone groups at the trypsin-NP interface revealed enhanced surface hydrophobicity after trypsin adsorption.

FIGS. 11A-11E Simulation results of IgG adsorption on to the 600-chain PPSU NP. (FIG. 11A) Atomistic simulation snapshots showing the adsorption of IgG (cyan) onto the surface of the 600-chain NP in three parallel simulations. Only one IgG molecule can be simulated in each system due to the limit set by space constraints. (FIG. 11B) Orientations of the 3 IgG molecules in the three parallel simulations are non-specific upon adsorption. The adsorption sites are colored red. (FIG. 11C) The Lennard-Jones IgG-NP interactions dominated over the IgG-NP Coulombic interactions, supporting the hydrophobicity-driven feature of IgG adsorption. (FIG. 11D) Percentage of sulfone groups at the IgG-NP contact region revealing enhanced NP surface hydrophobicity after IgG adsorption. (FIG. 11E) No significant (ns) differences in IgG hydration were detected between the adsorbed IgG and unbound IgG. The numbers of IgG-water H-bonds and of water neighbors of IgG were calculated. Error bars represent standard deviations. Statistical significance is determined by Tukey-post hoc test.

FIGS. 12A-12B Simulation results of BSA adsorption on to the 600-chain PPSU NP. (FIG. 12A) Atomistic simulation snapshot showing the adsorption of BSA (cyan) onto the surface of the 600-chain NP. (FIG. 12B) Quantitative calculation of the surface ratio of sulfone groups at the BSA-NP interface (within 2.5 Å of the BSA) revealing enhanced surface hydrophobicity after BSA adsorption.

FIGS. 13A-13H PPSU NPs preserve protein function within stable adlayers. (FIG. 13A) Schematic illustration of the rapid and facile process of coating PP SU NPs with protein adlayers. After incubation, unbound proteins are removed by thorough washing. (FIG. 13B) The zeta potential of protein-coated NPs is dependent on the adsorbed proteins. (FIG. 13C) SAXS data supporting the increase of shell thicknesses from 5.3 nm to 7.1 nm after BSA adsorption. (FIG. 13D) Trypsin is stably adsorbed at NP surfaces and free trypsin is undetectable by MALDI-TOF mass spectrometry in the supernatant after centrifugation of trypsin@NP. Samples were stored at 4° C. for 48 h prior to centrifugation. (FIG. 13E) Kinetic assay demonstrating bioactivity of adsorbed trypsin. Trypsin is encapsulated within the NPs for comparison. (FIG. 13F) Fluorescence of GFP was detectable for GFP@NP but not for GFP-encapsulated NPs. (FIG. 13G) Immunogold labelling showing the binding of pre-adsorbed anti-CD4 antibodies to secondary antibodies. 10 nm colloidal gold-secondary antibody, indicated by the arrows. (FIG. 13H) Targeting of irreversibly adsorbed anti-CD3 towards Jurkat T cells is confirmed by flow cytometry as assessed by percentage of NP positive cells and median fluorescence intensity (MFI). Statistical significance is determined by Tukey-post hoc test: *p<0.005, **p<0.001, ***p<0.0005, ****p<0.0001.

FIGS. 14A-14B Preparation of protein-coated NPs with improved colloidal stability in PBS. (FIG. 14A) PPSU NPs (5 mg/mL) were suspended in protein solution (10 mg/mL in PBS) or pooled human plasma for 5 min, followed by centrifugation and extensive washing to remove dynamically adsorbed proteins as well as residual proteins in solution. Output of protein-coated NPs (normalized to uncoated NPs in water) was measured by nanoparticle tracking analysis after extrusion (200 nm). Cluster formation of PPSU NPs in PBS without protein is included for comparison. (FIG. 14B) DLS size distribution analysis of 1 mg/mL of PPSU NPs in PBS with different concentrations of BSA.

FIGS. 15A-15B Imaging BSA-coated NPs by TEM and cryo-STEM. (FIG. 15A) A representative TEM image showing BSA@NP in water with 10 mg/mL of BSA. (FIG. 15B)

Simultaneously recorded cryo-STEM (left) and cryo-SEM (right) images showing BSA@NP and its surface in PBS. Scale bar in inset, 50 nm.

FIG. 16 Undetectable fluorescence in the supernatant confirming no desorption of FITC-BSA from FITC-BSA@NP. The sample of FITC-BSA@NP was incubated in pooled human plasma at 37° C. for 48 h prior to centrifugation.

FIG. 17 Schematic illustration of the fluorescence-based kinetic assay for exploring the activity of irreversibly adsorbed trypsin (trypsin@NP). The fluorescence of FITC is self-quenched on BSA and will be recovered upon trypsin proteolysis.

FIG. 18 Schematic illustration of the rapid and facile process in targeting PPSU NPs using pre-adsorbed antibodies as targeting moieties. After the two-step mixing in PBS, unbound proteins were removed by thorough washing of the NPs with PBS.

FIGS. 19A-19B Immunogold labelling. (FIG. 19A) Anti-CD4 antibodies and BSA co-adsorbed NPs stained with secondary gold-coupled antibodies. (FIG. 19B) BSA adsorbed NPs stained with secondary gold-coupled antibodies. All the 10 nm colloidal gold was adsorbed at NP surfaces in (FIG. 19A), whereas unbound colloidal gold was observed in (FIG. 19B). Both the samples contained 1 wt % of unbound BSA.

FIGS. 20A-20E Optimizing nanotherapy to inhibit anaphylaxis in a humanized mouse model. (FIG. 20A) PPSU-based nanomedicines consisting of co-adsorbed anti-Siglec-6 and anti-FcεRIα with controlled surface density (as wt % of PPSU) of anti-Siglec-6. (FIG. 20B) Imaging the nanomedicines (blue, indicated by red arrows) on the surface of a mast cell (grey) by SEM. (FIG. 20C) Activation of mast cells (left) is achieved via cross-linking of FcεRIα by anti-FcεRIα/BSA@NP, whereas the nanomedicine inhibits mast cell degranulation (right) via co-localized engagement. (FIG. 20D) Optimizing nanomedicine formulation via adjusting the surface density of anti-Siglec-6. In vitro results showing that lower anti-Siglec-6 density is more effective in suppressing CD107a and CD63 expression. (FIG. 20E) In vitro results demonstrating the importance of binding Siglec-6 in close proximity and time with engagement of FcεRI in suppressing CD107a and CD63 expression by mast cells. The optimized formulation (0.01 wt % of anti-Siglec-6) is used for the nanomedicine; ctr-a: Anti-Siglec-6/BSA@NP+Anti-FcεRIα; ctr-b: Anti-Siglec-6+Anti-FcεRIα+BSA; ctr-c: BSA@NP+Anti-FcεRIα. Inhibition is normalized from mast cells receiving only anti-FcεRIα and expressing a positive population mean of 52.7±5% for CD63 and 71.2±8% for CD107a (n=3

FIGS. 21A-21B illustrate the effect of anti-Siglec-6 density on PPSU nanoparticles. FIG. 21A shows availability of Siglec-6 on the cell surface after treating cells with PPSU. FIG. 21B illustrates dimeric crosslinking with the Siglec-6 on the cell surface.

FIGS. 22A-22B. Optimizing nanotherapy to inhibit anaphylaxis in a humanized mouse model. (FIG. 22A) The optimize5d nanomedicine (2.5 wt % of anti-Siglec-6) succeeds in administering allergen immunotherapy without triggering anaphylaxis in a humanized mouse model. Results were from 2 combined datasets (total n=6). (FIG. 22B) Humanized mice (n=5) were IgE-sensitized for 4-hydroxy-3-nitrophenylacetyl hapten-BSA (HN-BSA) followed by intravenous injections of formulations containing different combinations of PPSU NP, HN-BSA, and anti-Siglec-6. Mice injected with solubilized or NP-bound HN-BSA experienced a decrease of 2-2.5° C. in body temperature, indicating the onset of anaphylaxis. In contrast, the presence of anti-Siglec-6 in conjunction with HN-BSA on the surface of NPs resulted in inhibition of anaphylaxis. Statistical significance is determined by Tukey-post hoc test: *p<0.05, **p<0.01, ***p<0.005, ****p<0.001.

FIGS. 23A-23C illustrates a temperature response analysis of the protective effects of PPSU-bound anti-Siglec-6 and anti-FcεRIα antibodies. Effects of aSig6/aFc PPSU after day 1 (FIG. 23A), day 3 (FIG. 23B), and day 6 (FIG. 23C), are shown.

FIGS. 24A-24C illustrates a clinical score analysis of the protective effects of PPSU-bound anti-Siglec-6 and anti-FcεRIα antibodies. Effects of aSig6/a Fc PPSU after day 1 (FIG. 24A), day 3 (FIG. 24B), and day 6 (FIG. 24C), are shown.

FIG. 25 TNF-α release measured after incubating human skin mast cells with test formulations for 3 h (n=4). Adsorbed anti-FcεRIα antibodies remained functional, and secretion was inhibited by co-localized engagement of both FcεRIα and Siglec-6 receptors. Statistical significance was determined by Tukey-post hoc test: *p<0.05, **p<0.01, ***p<0.005, **** p<0.001.

FIGS. 26A-26B Representative SEM images showing a mast cell treated with PBS (FIG. 26A) or the nanomedicine of anti-FcεRIα/anti-Siglec-6 co-adsorbed NPs (FIG. 26B). The NPs are indicated by the red arrows.

FIG. 27 Process of in vivo testing. Design of humanized mast cell mouse anaphylaxis protocol adapted from Dispenza et al⁹.

FIG. 28 Inhibition of degranulation in human skin derived mast cells. CD63 is a key marker of degranulation in mast cells and was used here to indicate the degree of degranulation in a mast cell population. Lower anti-Siglec-6 density improved inhibitory effects of PPSU HNs co-loaded with HN-BSA antigen and anti-Siglec-6 antibody. Mast cells were pre-treated with 50 ng of anti-HN chimeric human IgE for 3 hours before subsequent incubation with test formulations containing 100 ng of HN-BSA (0.25 wt %) and variable amounts of anti-Siglec-6 adsorbed on PPSU HNs. Interestingly, a lack of co-localized delivery had a stronger impact when utilizing HN-BSA as compared to anti-FcεRIα (FIG. 20E). This may be due in part to the additional requirement for IgE engagement in this model. * P<0.05, n=2; one-way ANOVA with Tukey's multiple comparisons.

### DETAILED DESCRIPTION OF THE INVENTION

The present invention has been described in terms of one or more preferred embodiments, and it should be appreciated that many equivalents, alternatives, variations, and modifications, aside from those expressly stated, are possible and within the scope of the invention.

The present disclosure provides methods of making protein-coated poly(propylene sulfone) (PPSU) homopolymer nanoparticles. The methods may be used to prepare compositions of such nanoparticles coated with Siglec-6 and FcεRI antibodies for targeting mast cells. The compositions may inhibit and/or reduce mast cell secretion including degranulation, and thus, may also inhibit allergic reactions.

In a first aspect, provided herein is a method for adsorbing at least one protein onto the surface of a homopolymer nanoparticle, the method comprising incubating the nanoparticle with the at least one protein in a buffer at room temperature. In preferred embodiments, the nanoparticle is a PPSU nanoparticle. The concentration of the at least one protein may be higher than the concentration of the nanoparticle. The concentration of the at least one protein may be at least twice as high as the concentration of the nanoparticle in the buffer. The method may comprise adsorbing at least two proteins to the nanoparticle. The protein may be incubated at between about 0.001 wt % and about 1000 wt % of the total mass of the PPSU nanoparticle.

As used herein, the terms “to adsorb” and “to coat” generally means covering, or surface encapsulating, target molecules.

As used herein, the term “nanoparticle” refers to a structure that ranges in size between about 1 nm to about 200 nm and any size in-between (e.g., between 10 nm and 200 nm, between 1 nm and 100 nm, between 1 nm and 40 nm, between 1 nm and 30 nm, between 1 nm and 20 nm, between 1 nm and 15 nm, between 100 nm and 200 nm, and between 150 nm and 200 nm). The nanoparticle used herein may comprising synthetic self-assembling homopolymer poly(propylene sulfone) (PPSU)⁴. The PPSU may have any number of repeat units, for example, from 10 to 100 repeat units, especially for example 20 repeat units. In exemplary embodiments, the nanoparticle comprises PPSU₂₀. Structures of PPSU nanoparticles are described in U.S. patent application Ser. No. 16/942,228, the contents of which are incorporated by reference in its entirety.

The nanoparticle may possess a negatively charged surface that promotes attachment of positively charged regions on proteins to the nanoparticle. In some embodiments, the surface zeta potential in water of the nanoparticle is in the range of about −100 mV to about +100 mV, and all values in-between, including about 45 mV.

The proteins may be placed in a sterile container together at the desired ratios and/or concentrations prior to subsequent addition of nanoparticles to achieve a mixture of proteins and nanoparticles consistent with desired weight percentages. Contact between the proteins and the nanoparticles results in adsorption of the proteins on the nanoparticle surfaces due to physical and electrostatic interactions without the need for covalent binding.

The nanoparticle may be incubated with the proteins for at least one second. The incubation may be performed between about 1 second and about 7 minutes, or for any range or duration in between. For example, the incubation may be performed for between about 1 second and about 5 minutes, or between about 3 minutes and 7 about minutes. In exemplary embodiments, the nanoparticle and proteins are incubated for about 5 minutes.

The nanoparticle may be incubated with the proteins at a temperature above freezing and below boiling of the particles and proteins in solution. For example, the incubation may be performed at between 2° C.-100° C. In exemplary embodiments, the incubation is performed at about room temperature, or between about 20° C. and about 22° C.

After adsorbing the protein cargo, the nanoparticle may be incubated with a capping or filler agent, such as albumin. The albumin may comprise bovine serum albumin (BSA). The nanoparticle may be incubated with the BSA for between about 3 and about 7 minutes at room temperature. The nanoparticle may be incubated with the capping agent at a minimum of a 1:1 molar ratio of nanoparticle: capping agent.

The method may further comprise washing the nanoparticle with the buffer two remove excess protein. The nanoparticle may be washed more than one time. The nanoparticle may be washed between 1 and 3 times. In exemplary embodiments, the nanoparticle is washed 3 times.

The protein adsorbed nanoparticle may be suspended and stored in the buffer.

In exemplary embodiments, the buffer comprises phosphate buffered saline (PBS). However, any standard buffer may be used, such as IVIES, a Tris buffer, ADA, ACES, PIPES, MOPS, HEPES, etc. A non-exhaustive list of suitable buffers is found at https://www.sigmaaldrich.com/US/en/technical-documents/protocol/protein-biology/protein-concentration-and-buffer-exchange/buffer-reference-center, which is incorporated herein by reference in its entirety.

In an embodiment, the at least one protein adsorbed to the nanoparticle is an antibody. At least two antibodies may be adsorbed. In an exemplary embodiment, the method comprises adsorbing an anti-Siglec-6 antibody and an anti-FcεRI antibody. The anti-Siglec-6 antibody may be antibody clone 767329 from R&D Systems. The anti-FcεRI antibody may be antibody clone AER-37 (CRA1) from Fisher Scientific. The inventors show that anti-Siglec-6 and anti-FcεRI coated nanoparticles bind to Siglec-6 and FcεRI on mast cell surfaces, and reduce or inhibit mass cell degranulation and activation.

Siglecs (sialic acid-binding immunoglobulin-like lectins) are cell surface receptors that bind sialic acid. They are found primarily on the surface of immune cells and are a subset of the I-type lectins. Engagement of CD33-family sialic acid-binding immunoglobulin-like lectin (Siglec) receptors are shown to have inhibitory effects on various cells including mast cells^{1,2}. Of the siglec family of proteins that are expressed on mast cells (Siglec-2/CD22, Siglec-3/CD33, Siglec-5, Siglec-6, Siglec-8, and Siglec-10), Siglec-6 is the most specific³. Sialic acid-binding Ig-like lectin 6 (Siglec-6) is a protein encoded by the SIGLEC6 gene. Siglec-6 is also called CD33-like, OB [leptin]-binding protein 1 (OB-BP1) and CD327. The full-length sequence of Siglec-6 is shown in SEQ ID NO: 1. Siglec-6 is expressed on a variety of cell types, including placenta, skeletal and smooth muscle, pancreatic ductal cells, gallbladder, appendix, lung and almost all hematopoietic cells including mast cells. Antibody ligation of Siglec-6 receptors on human CD34+progenitor-derived mast cells has previously been shown to inhibit GM-CSF secretion and slightly reduce degranulation in response to IgE crosslinking⁸. More potent inhibition has been observed by co-crosslinking Siglec-6 and FcεRI⁹.

The IgE receptor, also known as FcεRI, or Fc epsilon RI, is the high-affinity receptor for the Fc region of immunoglobulin E (IgE), an antibody isotype involved in the immune response against allergy disorders and parasite infections. FcεRI is found on epidermal Langerhans cells, eosinophils, basophils, and mast cells. It is a tetrameric receptor complex that binds the Fc portion of the c heavy chain of IgE. It consists of one alpha (FcεRIα—antibody binding site), one beta (FcεRIβ—which amplifies the downstream signal), and two gamma chains (FcεRIγ—the site where the downstream signal initiates) connected by two disulfide bridges. The full-length sequence of FcεRI is shown in SEQ ID NO: 2. Crosslinking of the FcεRI via IgE-antigen complexes leads to degranulation of mast cells and release of inflammatory mediators¹⁰. However, as discussed above, co-crosslinking of Siglec-6 and FcεRI yields inhibition of degranulation from mast cells.

The anti-Siglec-6 the anti-FcεRIα antibodies may be incubated with the nanoparticles at between about 0.0001 wt % and 10000 wt % PPSU or any wt % and range in between, e.g. between about 0.001 wt % and about 1000 wt % PPSU.

In exemplary embodiments, the desired surface density of the anti-Siglec-6 antibodies on the PPSU surface is between about 0.01 wt % and about 10 wt % PPSU, or about 0.01 wt %, about 0.1 wt %, about 1 wt %, or about 2.5 wt %. In exemplary embodiments, the desired surface density of the anti-FcεRIα antibodies on the PPSU surface is between about 0.01 wt % and about 1 wt %, or any wt % and range in between, such as about 0.1 wt % or about 0.2 wt % PPSU. In exemplary embodiments, PPSU nanoparticles have 0.2 wt % anti-FcεRIα antibody surface density and 2.5 wt % anti-Siglec-6 antibody surface density. The “surface density”, as used herein, is the density of antibody/protein on the surface of the nanoparticle.

The anti-Siglec-6 antibodies and the anti-FcεRIα antibodies may be placed in a sterile container at the desired ratio and concentration prior to subsequent addition of nanoparticles to achieve a mixture of antibodies and nanoparticles consistent with the weight percentages listed above. Contact between antibodies and PPSU nanoparticles results in adsorption of antibodies on PPSU nanoparticle surfaces due to physical and electrostatic interactions without the need for covalent binding.

The anti-Siglec-6 antibody may target Siglec-6 receptor (SEQ ID NO: 1). The anti-FcεRIα antibody may target FcεRIα subunit (SEQ ID NO: 2).

In a second aspect, provided herein is a composition for reducing mass cell secretion, the composition comprising a PPSU homopolymer nanoparticle coated with at least one of an anti-Siglec-6 antibody and an anti-FcεRI antibody. The nanoparticle may comprise both an anti-Siglec-6 antibody and an anti-FcεRI antibody. The composition may further comprise a pharmaceutically acceptable excipient carrier or diluent. The exact nature of the carrier, excipient or diluent will depend upon the desired use for the composition, and may range from being suitable or acceptable for veterinary uses to being suitable or acceptable for human use. The composition may optionally include one or more additional compounds.

In a third aspect, provided herein is a method for treating an allergic reaction in a subject in need thereof. The allergic reaction may be a type I hypersensitivity reaction, such as anaphylaxis. The method comprises administering to the subject a therapeutically effective amount of a PPSU homopolymer nanoparticle coated with at leas tone of an anti-Siglec-6 antibody and an anti-FcεRI antibody or a composition comprising the nanoparticle. As used herein, a “subject” may be interchangeable with “patient” or “individual,” and means an animal, which may be a human or non-human animal, in need of treatment. In particular embodiments, the subject is a human subject. A “subject in need of treatment” may include a subject having a disease, disorder, or condition that may be characterized as an allergic reaction, type I hypersensitivity, or anaphylaxis.

As used herein, the terms “treating” and “to treat” mean to alleviate symptoms, eliminate the causation of resultant symptoms either on a temporary or permanent basis, and/or to prevent or slow the appearance or to reverse the progression or severity of resultant symptoms of the named disease or disorder. The subject may be responsive to therapy with one or more of the nanoparticles disclosed herein in combination with one or more additional therapeutic agents. The term “treating” thus includes reducing or inhibiting activation of mast cells, including, secretion and degranulation. As used herein the term “inhibit” refers to a reduction in activity or progression.

The compositions and nanostructures described herein can be used to treat allergic diseases and type I hypersensitive reactions, including, but not limited to, for example, hypersensitivity, allergic reactions, anaphylaxis, allergic rhinitis, food allergy, atopic dermatitis, and allergic asthma. Anaphylactic degranulation, also known as mast cell degranulation or mast cell secretion, includes the immediate and explosive release of pharmacologically active pre-formed mediators from storage granules and concurrent synthesis of inflammatory lipid mediators from arachidonic acid; some of these mediators include histamine, leukotriene (LTC₄ and LTD₄ and LTB₄), GM-CSF, TNFα, IL-3, IL-4, IL-5, IL-6, IL-10, and IL-13, chemokines including CCL2, CCL3, CCL5, and CXCL8, and growth factors such as SCF, FGF, VEGF, angiogenin, and prostaglandin, which act on proteins (e.g., G-protein coupled receptors) located on surrounding tissues. Symptoms of allergic diseases that may be treated include the reduction in one or more symptom associated with the allergic diseases, including mast cell degranulation, vasodilation and smooth-muscle contraction.

As used herein the term “effective amount” refers to the amount or dose of the nanoparticle that provides the desired effect. The effective amount may the amount or dose of the nanoparticle, upon single or multiple dose administration to the subject, which provides the desired effect in the subject under diagnosis or treatment. The desired effect may be reducing the effect of or symptoms of anaphylaxis or type I hypersensitivity.

An effective amount can be readily determined by those of skill in the art, including an attending diagnostician, by the use of known techniques and by observing results obtained under analogous circumstances. In determining the effective amount or dose of compound administered, a number of factors can be considered by the attending diagnostician, such as: the species of the subject; its size, age, and general health; the degree of involvement or the severity of the disease or disorder involved; the response of the individual subject; the particular compound administered; the mode of administration; the bioavailability characteristics of the preparation administered; the dose regimen selected; the use of concomitant medication; and other relevant circumstances.

When used to treat or prevent a disease or symptoms of a disease, such as an allergic reaction, the compositions described herein may be administered singly, as nanoparticles or in mixture or combination with other agents (e.g., therapeutic agents) useful for treating such diseases and/or the symptoms associated with such diseases. Such agents may include, but are not limited to, epinephrine, antihistamines, cortisone, or beta-agonists to name a few. The nanoparticles may be administered in the form of nanoparticles per se, or as pharmaceutical compositions comprising the nanoparticles.

Pharmaceutical compositions comprising the nanoparticles may be manufactured by means of conventional mixing, dissolving, granulating, dragee-making, levigating, emulsifying, encapsulating, entrapping or lyophilization processes. The compositions may be formulated in a conventional manner using one or more physiologically acceptable carriers, diluents, excipients or auxiliaries which facilitate processing of the compounds into preparations which can be used pharmaceutically.

Pharmaceutical compositions may take a form suitable for virtually any mode of administration, including, for example, topical, ocular, oral, buccal, systemic, nasal, injection, transdermal, rectal, vaginal, etc., or a form suitable for administration by inhalation or insufflation. For topical administration, the compound(s) may be formulated as solutions, gels,

ointments, creams, suspensions, etc. as are well-known in the art. Systemic formulations include those designed for administration by injection, e.g., subcutaneous, intravenous, intramuscular, intrathecal or intraperitoneal injection, as well as those designed for transdermal, transmucosal oral or pulmonary administration.

Useful injectable preparations include sterile suspensions, solutions or emulsions of the active compound(s) in aqueous or oily vehicles. The compositions may also contain formulating agents, such as suspending, stabilizing and/or dispersing agent. The formulations for injection may be presented in unit dosage form, e.g., in ampules or in multidose containers, and may contain added preservatives. Alternatively, the injectable formulation may be provided in powder form for reconstitution with a suitable vehicle, including but not limited to sterile pyrogen free water, buffer, dextrose solution, etc., before use. To this end, the active compound(s) may be dried by any art-known technique, such as lyophilization, and reconstituted prior to use.

For transmucosal administration, penetrants appropriate to the barrier to be permeated are used in the formulation. Such penetrants are known in the art.

Preparations for oral administration may be suitably formulated to give controlled release of the compound, as is well known. For buccal administration, the compositions may take the form of tablets or lozenges formulated in conventional manner. For rectal and vaginal routes of administration, the compound(s) may be formulated as solutions (for retention enemas) suppositories or ointments containing conventional suppository bases such as cocoa butter or other glycerides.

For nasal administration or administration by inhalation or insufflation, the compound(s) can be conveniently delivered in the form of an aerosol spray from pressurized packs or a nebulizer with the use of a suitable propellant, e.g., dichlorodifluoromethane, trichlorofluoromethane, dichlorotetrafluoroethane, fluorocarbons, carbon dioxide or other suitable gas. In the case of a pressurized aerosol, the dosage unit may be determined by providing a valve to deliver a metered amount. Capsules and cartridges for use in an inhaler or insufflator (for example capsules and cartridges comprised of gelatin) may be formulated containing a powder mix of the compound and a suitable powder base such as lactose or starch.

For ocular administration, the compound(s) may be formulated as a solution, emulsion, suspension, etc. suitable for administration to the eye. A variety of vehicles suitable for administering compounds to the eye are known in the art.

For prolonged delivery, the compound(s) can be formulated as a depot preparation for administration by implantation or intramuscular injection. The compound(s) may be formulated with suitable polymeric or hydrophobic materials (e.g., as an emulsion in an acceptable oil) or ion exchange resins, or as sparingly soluble derivatives, e.g., as a sparingly soluble salt.

Alternatively, transdermal delivery systems manufactured as an adhesive disc or patch which slowly releases the compound(s) for percutaneous absorption may be used. To this end, permeation enhancers may be used to facilitate transdermal penetration of the compound(s).

Alternatively, other pharmaceutical delivery systems may be employed. Liposomes and emulsions are well-known examples of delivery vehicles that may be used to deliver compound(s).

Certain organic solvents such as dimethyl sulfoxide (DMSO) may also be employed, although usually at the cost of greater toxicity.

The pharmaceutical compositions may, if desired, be presented in a pack or dispenser device which may contain one or more unit dosage forms containing the compound(s). The pack may, for example, comprise metal or plastic foil, such as a blister pack. The pack or dispenser device may be accompanied by instructions for administration.

Determination of an effective dosage for a particular use and mode of administration is well within the capabilities of those skilled in the art. Effective dosages may be estimated initially from in vitro activity and metabolism assays. For example, an initial dosage for use in animals may be formulated to achieve a circulating blood or serum concentration of the composition that is at or above an IC₅₀ of the particular composition as measured in an in vitro assay. Calculating dosages to achieve such circulating blood or serum concentrations taking into account the bioavailability of the particular composition via the desired route of administration is well within the capabilities of skilled artisans. Initial dosages can also be estimated from in vivo data, such as animal models. Animal models useful for testing the efficacy of the active metabolites to treat or prevent the various diseases described above are well-known in the art. Animal models suitable for testing the bioavailability and/or metabolism of compositions are also well-known. Ordinarily skilled artisans can routinely adapt such information to determine dosages suitable for human administration.

Dosage amounts will typically be in the range of from about 0.0001 mg/kg/day, 0.001 mg/kg/day or 0.01 mg/kg/day to about 100 mg/kg/day, but may be higher or lower, depending upon, among other factors, the activity of the active composition, the bioavailability of the composition, its metabolism kinetics and other pharmacokinetic properties, the mode of administration and various other factors, discussed above. Dosage amount and interval may be adjusted individually to provide plasma levels which are sufficient to maintain therapeutic or prophylactic effect. For example, the compositions may be administered once per week, several times per week (e.g., every other day), once per day or multiple times per day, depending upon, among other things, the mode of administration, the specific indication being treated and the judgment of the prescribing physician. In cases of local administration or selective uptake, such as local topical administration, the effective local concentration of compositions may not be related to plasma concentration. Skilled artisans will be able to optimize effective dosages without undue experimentation.

Miscellaneous

Unless otherwise specified or indicated by context, the terms “a”, “an”, and “the” mean “one or more.” For example, “a molecule” should be interpreted to mean “one or more molecules.”

As used herein, “about”, “approximately,” “substantially,” and “significantly” will be understood by persons of ordinary skill in the art and will vary to some extent on the context in which they are used. If there are uses of the term which are not clear to persons of ordinary skill in the art given the context in which it is used, “about” and “approximately” will mean plus or minus ≤10% of the particular term and “substantially” and “significantly” will mean plus or minus >10% of the particular term.

As used herein, the terms “include” and “including” have the same meaning as the terms “comprise” and “comprising.” The terms “comprise” and “comprising” should be interpreted as being “open” transitional terms that permit the inclusion of additional components further to those components recited in the claims. The terms “consist” and “consisting of” should be interpreted as being “closed” transitional terms that do not permit the inclusion additional components other than the components recited in the claims. The term “consisting essentially of” should be interpreted to be partially closed and allowing the inclusion only of additional components that do not fundamentally alter the nature of the claimed subject matter.

All methods described herein can be performed in any suitable order unless otherwise indicated herein or otherwise clearly contradicted by context. The use of any and all examples, or exemplary language (e.g., “such as”) provided herein, is intended merely to better illuminate the invention and does not pose a limitation on the scope of the invention unless otherwise claimed. No language in the specification should be construed as indicating any non-claimed element as essential to the practice of the invention.

All references, including publications, patent applications, and patents, cited herein are hereby incorporated by reference to the same extent as if each reference were individually and specifically indicated to be incorporated by reference and were set forth in its entirety herein.

Preferred aspects of this invention are described herein, including the best mode known to the inventors for carrying out the invention. These aspects and embodiments are illustrative and non-exhaustive in nature. Variations of those preferred aspects may become apparent to those of ordinary skill in the art upon reading the foregoing description. The inventors expect a person having ordinary skill in the art to employ such variations as appropriate, and the inventors intend for the invention to be practiced otherwise than as specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

### REFERENCES

1. Duan et al. CD33 recruitment inhibits IgE-mediated anaphylaxis and desensitizes mast cells to allergen. J Clin Invest, 129(3). 1 Mar. 2019
2. Duan et al. Nanoparticles Displaying Allergen and Siglec-8 Ligands Suppress IgE-FcεRI-Mediated Anaphylaxis and Desensitize Mast Cells to Subsequent Antigen Challenge. J Immunol. ji1901212. 28 Apr. 2021.
3. Plum et al. Human Mast Cell Proteome Reveals Unique Lineage, Putative Functions, and Structural Basis for Cell Ablation. Immunity, 52(2). 18 Feb. 2020
4. Yu et al. Functional Inhibitory Siglec-6 Is Upregulated in Human Colorectal Cancer-Associated Mast Cells. Front. Immunol., 20 Sep. 2018
5. Du et al. Homopolymer self-assembly of poly(propylene sulfone) hydrogels via dynamic noncovalent sulfone-sulfone bonding. Nat. Commun, Volume 11, Article number: 4896. 29 Sep. 2020
6. Dispenza et al. Bruton's tyrosine kinase inhibition effectively protects against human IgE-mediated anaphylaxis. J Clin Invest. 130(9):4759-4770. 4 Aug. 2020
7. Caslin et al. The Use of Human and Mouse Mast Cell and Basophil Cultures to Assess Type 2 Inflammation. Methods Mol Biol. 1799:81-92. 29 Jun. 2018

### EXAMPLES

### Example. Bioactive Multi-Protein Adsorption Enables Targeted Mast Cell Nanotherapy

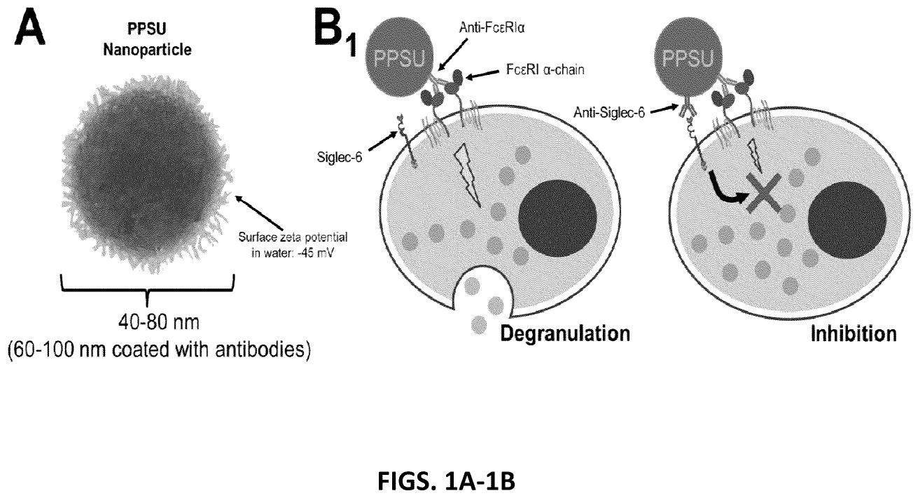

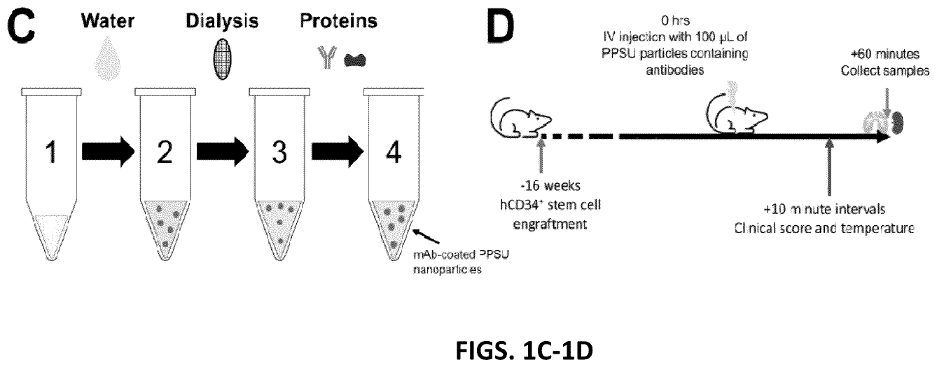

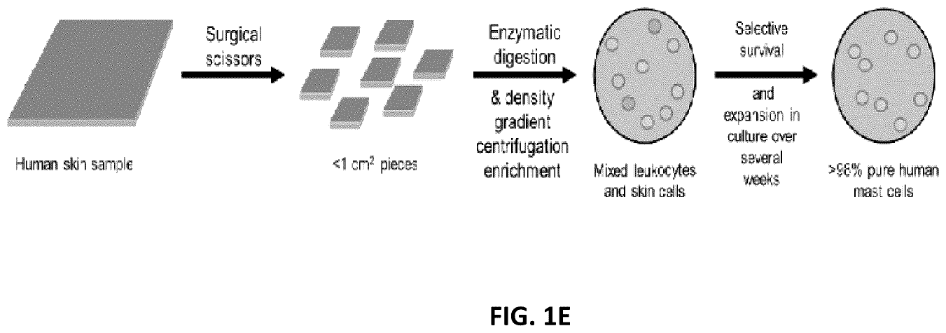

FIG. 1A is a fluidics model of the appearance of a spherical PPSU nanoparticle described herein. The nanoparticle possesses negatively charged surfaces composed of overlapping layers of polymer⁵. The nanoparticle has a surface zeta potential of ˜45 mV in water. The body of the nanoparticle is 40-80 nm in diameter without antibodies, and 60-100 nm in diameter after the antibodies are loaded. FIG. 1B provides a schematic of mast cell activation when exposed to the PPSU nanoparticle. As illustrated in the left panel, when PPSU is loaded with anti-FcεRIα antibodies only, degranulation occurs, and mediators are released, which will yield an allergic reaction. However, as shown in the right panel, when PPSU is loaded with both anti-FcεRIα antibodies and anti-Siglec-6 antibodies, degranulation is inhibited.

Proteins readily and often irreversibly adsorb to nanomaterial surfaces, resulting in denaturation and loss of bioactivity^{1,2}. Controlling this process to preserve protein structure and function has remained an elusive goal that would enhance the fabrication and biocompatibility of protein-based bioactive nanomaterials³⁻⁷. Here, we demonstrate that poly(propylene sulfone) (PPSU)⁸ nanoparticles support the controlled formation of multi-component enzyme and antibody coatings while maintaining their bioactivity. Simulations indicate that hydrophobic patches' on protein surfaces induce site-specific dipole relaxation on PPSU surfaces to noncovalently anchor proteins without disrupting hydrogen bonding or protein structure. As proof-of-concept, a nanotherapy for enhanced antibody-based targeting of mast cells and inhibition of anaphylaxis^{3,4} is demonstrated in a humanized mouse model. The ratio of co-adsorbed anti-Siglec-6^{10,11} and anti-FcεRIα antibodies is systematically optimized to effectively inhibit mast cell activation and degranulation. Protein immobilization on PPSU surfaces therefore provides a simple and rapid platform for the development of targeted nanomedicines.

Nanomaterials that preserve the bioactivity of anchored proteins hold promise for a wide range of applications as nanozymes, diagnostic sensors, and targeted delivery systems⁵⁻⁷. Although various synthetic platforms have been developed, unmodified nanoparticles (NPs) remain challenging in interfacing with a wide range of proteins without affecting protein folding or function^{7,12}. For example, NPs with hydrophobic surfaces tend to irreversibly adsorb protein adlayers, leading to denaturation and associated loss of protein bioactivity^{1,2}. It is therefore mandatory to involve hydrophilic “anti-fouling” polymers, such as poly(ethylene glycol) (PEG)¹³ and zwitterionic moieties^{14,15}, within NP surfaces to prevent this non-specific adsorption.

Given their highly hydrophilic surfaces after modifications, most NPs require the tedious process of chemical coupling to reliably interface with proteins to harness their bioactivity³⁻⁷. This method has simultaneously enabled the surface display of targeting antibodies but also limited the control to typically a single antibody type with minimal control over the surface density. Protein adsorption thus presents a comparatively facile noncovalent approach to rapidly engineer bioactive NP surfaces with multiple antibodies and proteins. However, application of this approach is hampered by the continued desorption or loss of activity of the protein layer¹⁶⁻¹⁸. Of note, controlling the composition and stability of anchored proteins, particularly for multiple antibodies, has remained challenging even when employing chemical coupling strategies, which have conjugation yields dependent on the reactivity of the NP surface and accessibility of binding sites within proteins.

We report on PPSU NPs that irreversibly adsorb nonspecific proteins without compromising protein function. This ready-to-use protein immobilization platform is effective for both single and multi-component antibody and enzyme coatings with well-controlled composition. To further demonstrate this design freedom, we engineer PPSU NPs into the first antibody-based nanomedicine for desensitizing mast cells (MCs) to allergen. The nanotherapy requires the controlled co-adsorption of two separate antibodies at an optimized ratio to tune the simultaneous signaling of Siglec-6 and FcεRIα receptors in close proximity on MC surfaces. This therapeutic strategy administers allergen immunotherapy without triggering anaphylaxis in a humanized mouse model.

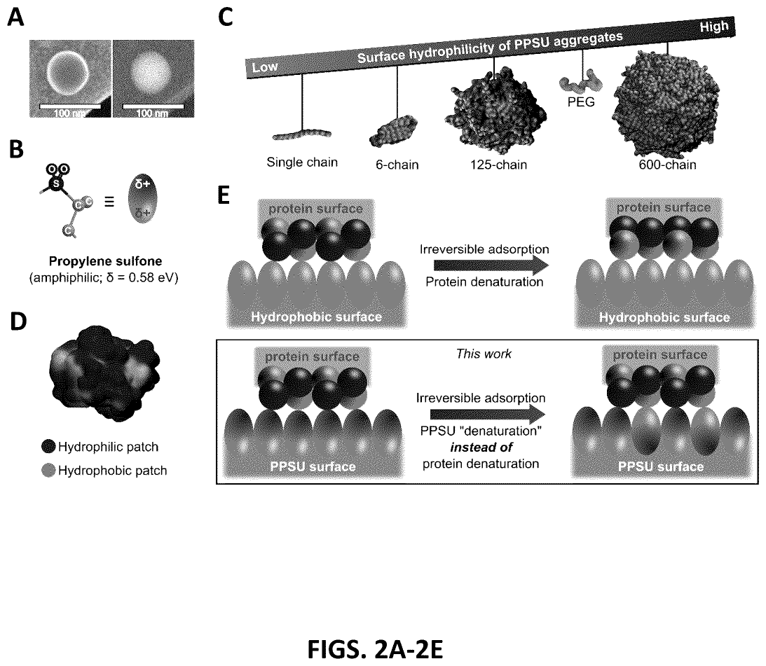

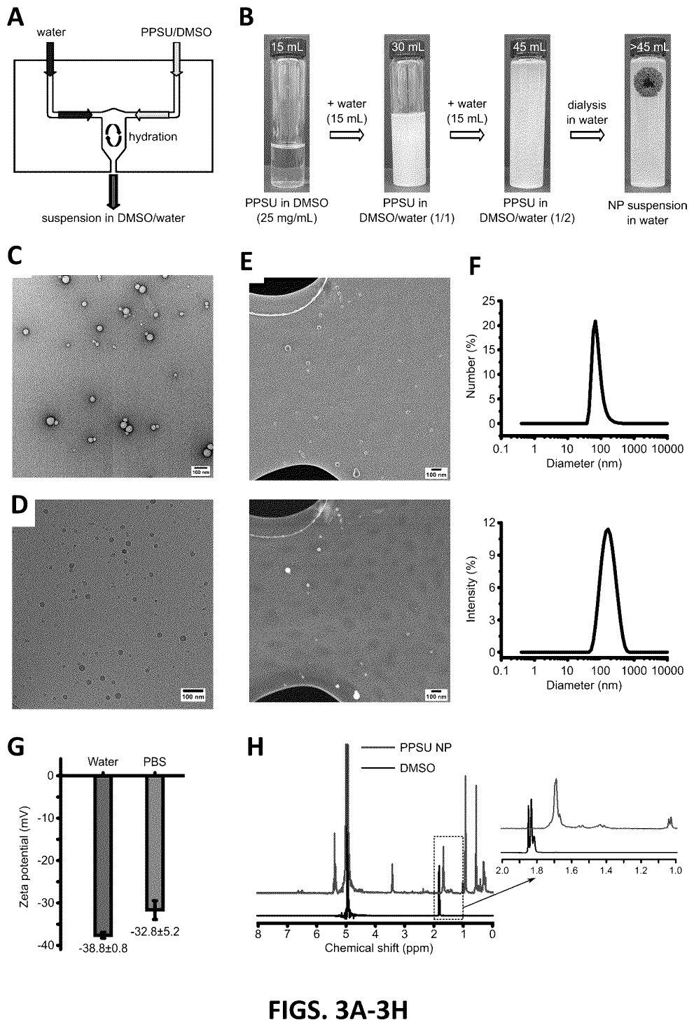

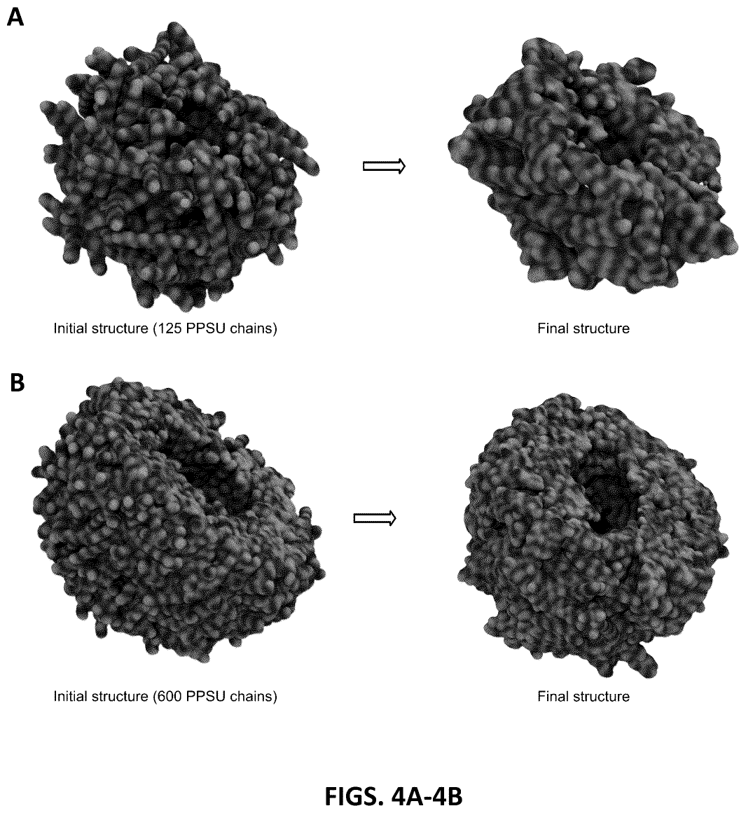

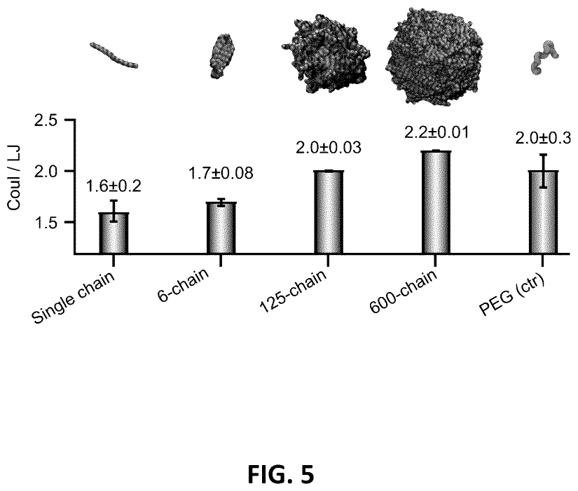

Hollow PPSU NPs (FIG. 2A) were fabricated at scale (FIG. 3) according to our previous report⁸. As prepared, the NPs are negatively charged on their surfaces with a zeta potential of <−30 mV in aqueous solution (FIG. 3). We ascribed this phenomenon to spontaneous orientation of propylene sulfone (PS, FIG. 2B) at the interface between water and PPSU due to inherent amphiphilicity within the repeating unit. To explore this postulate, explicit solvent all-atom molecular dynamics (AAMD) simulations of PPSU in water were performed to construct a hollow aggregate that mimics the experimentally observed PPSU NPs. Hollow NPs formed in both the 125-chain and 600-chain simulation systems (FIG. 2C) without severe deformation or collapse of shell structures (FIG. 4). Because PS orientation promotes the surface enrichment of polar sulfone groups, the NPs showed increased surface hydrophilicity when compared to a single PPSU chain (FIG. 2C). This was concluded by calculating the ratio of the Coulombic (polar) interactions between PPSU and water to the corresponding Lennard-Jones (nonpolar) interactions (FIG. 5). Note that the quantitative analysis of surface hydrophilicity has implicitly taken into account the morphology and size of aggregates¹⁹.

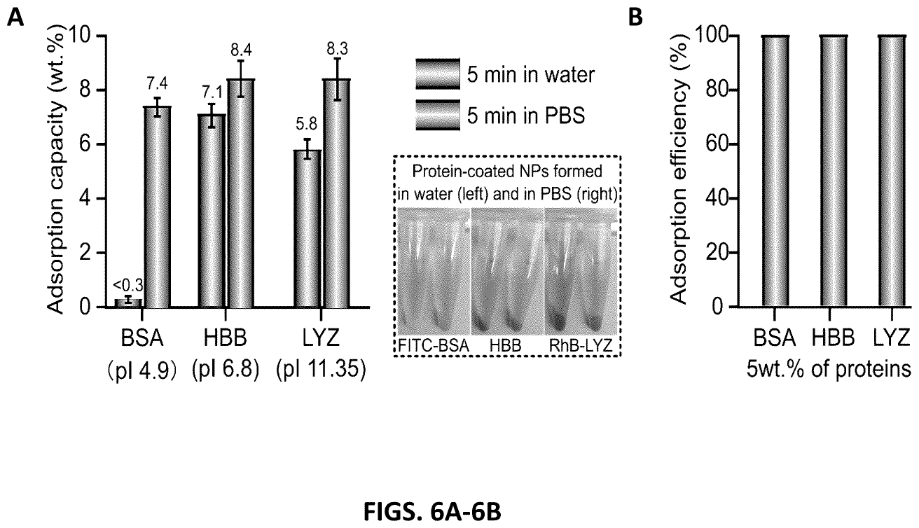

The simulations showed that the PPSU NPs exhibit comparable or even higher surface hydrophilicity compared to that of PEG. However, we were surprised to observe the rapid adsorption of nonspecific proteins by PPSU NPs in phosphate buffered saline (PBS) (FIG. 6). Protein surfaces are chemically diverse with characteristic distributions of surface accessible hydrophobic patches (FIG. 2D)⁹. Because the hydrophilic state of PPSU surfaces is induced by the aqueous solvent molecules, we inferred that the dominant orientations of interfacial PSs could also be directed by the hydration of adjacent patches experienced at protein interfaces. That is, a hydrophobic protein patch is likely to flip the interfacial PSs while a hydrated hydrophilic patch would preserve the hydrophilic state of PP SU surfaces (FIG. 2E). Such local hydrophilic/hydrophobic switching events thermodynamically favor the screening of electrostatic repulsion among sulfone groups, leading to small site-to-site heterogeneous NP surfaces at PPSU-protein interfaces.

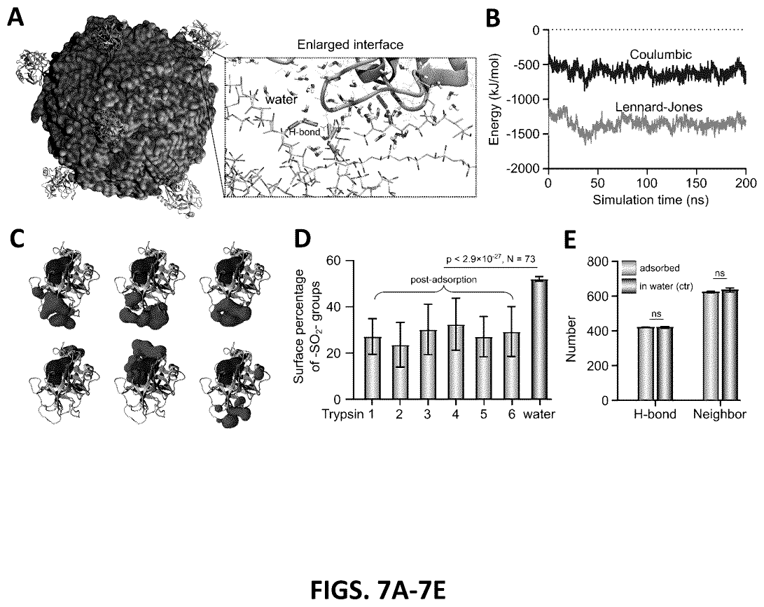

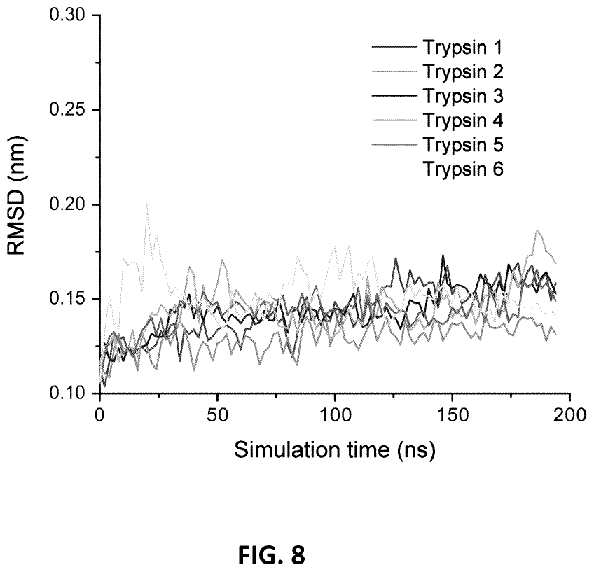

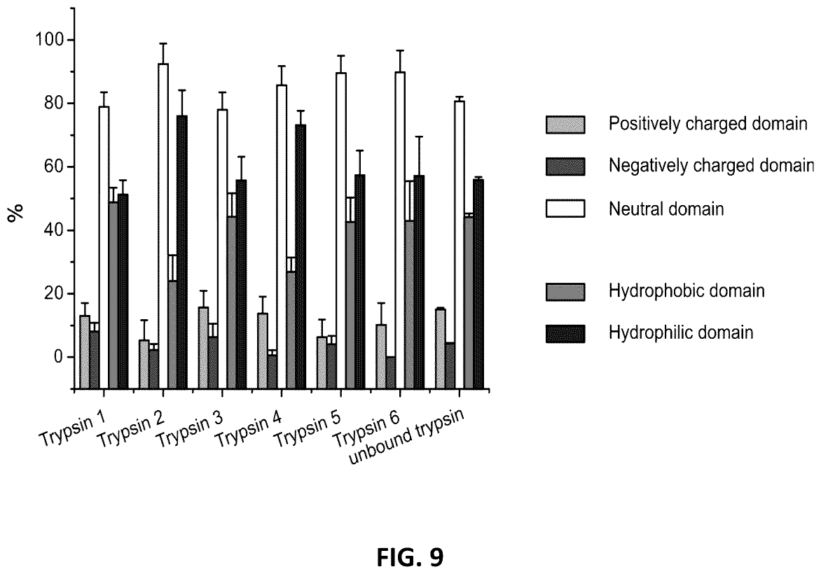

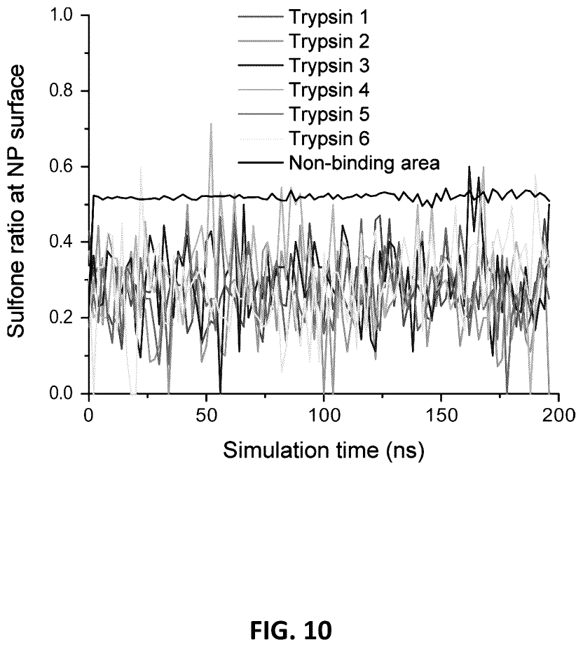

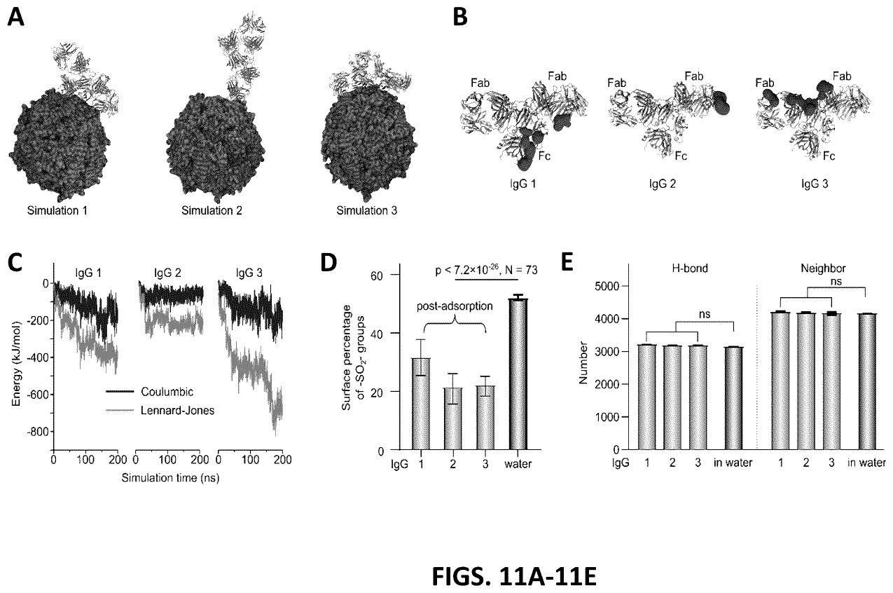

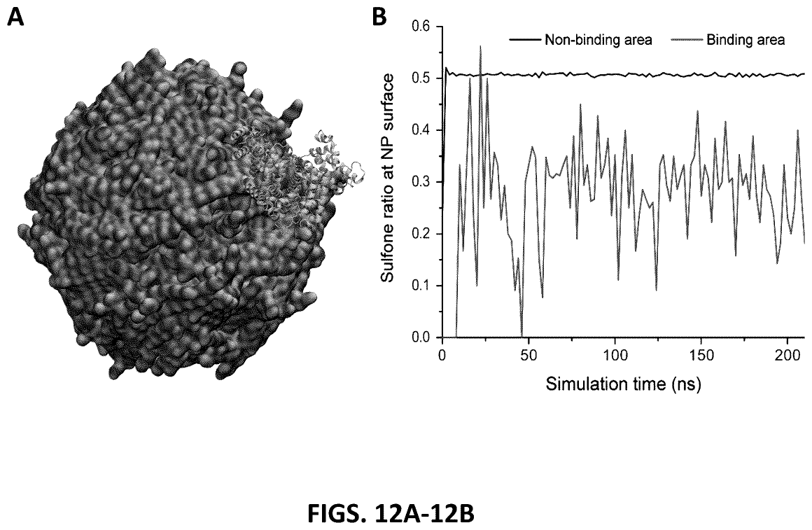

To investigate the mechanism of protein adsorption, the AAMD simulation of the 600-chain PPSU NP was proceeded by adding 6 molecules (the limit set by space constraints) of trypsin in the aqueous system. The last simulation snapshot is shown in FIG. 7A. All six trypsin molecules were adsorbed by the PPSU NP. Calculations of trypsin-NP interactions explicitly supported that the hydrophobic interactions were a major determinant of the adsorption process (FIG. 7B). The 3D structure of adsorbed trypsin was preserved at the PPSU surface, supported by the small root-mean-square deviation of approximately 1.5 Å for each trypsin molecule during the simulation (FIG. 8)²⁰. This is in contrast with traditional hydrophobic surfaces where proteins were often found to spread and unfold upon hydrophobic binding (FIG. 2E)². Furthermore, the 6 trypsin molecules were randomly oriented after anchoring (FIG. 7C and FIG. 9). The non-specific orientation of trypsin allowed the exposure of their active sites for 5 out of the 6 molecules, demonstrating retention of enzymatic activity even when shielding by further corona formation was taken into account¹⁶. Dipole relaxation of the PPSU backbone at all 6 PPSU-trypsin interfaces was confirmed via calculating the surface percentage of sulfone groups, revealing significantly enhanced surface hydrophobicity triggered by trypsin adsorption (FIG. 7D and FIG. 10). Simulations also showed that water lubricated the PPSU-trypsin contact regime via PPSU-water-trypsin H-bonds (FIG. 7A, inset). Furthermore, the hydration of trypsin, described by the amounts of trypsin-water H-bonds and water neighbors, remained unchanged despite hydrophobic adsorption (FIG. 7E). This retention of protein hydration is an appealing feature for hydrophilic surfaces and essential for preserving the bioactivity of proteins²⁰. Taken together, the simulation results suggested that restructuring of the PPSU surface, rather than denaturing the adsorbed trypsin, led to controlled interfacial hydrophobic interactions for preserving protein structure. Of note, similar results were reported by simulating the PP SU NP with immunoglobulin G (IgG) (FIG. 11) in a bovine serum albumin (BSA) solution (FIG. 12).

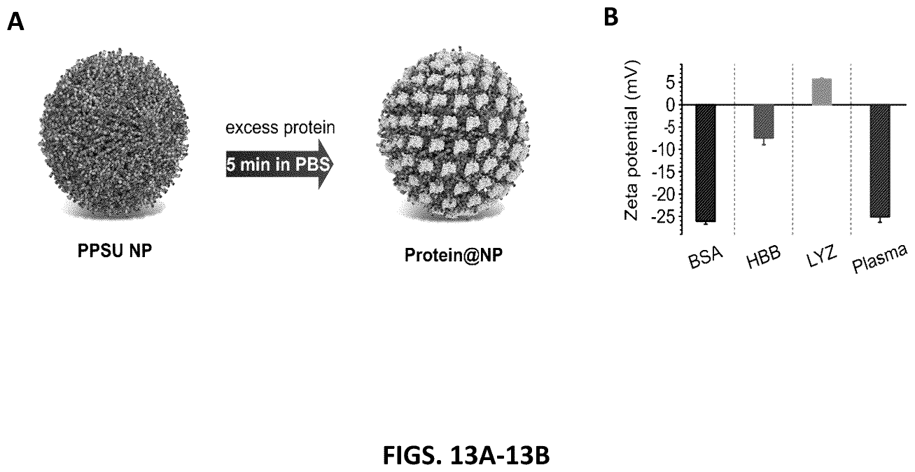

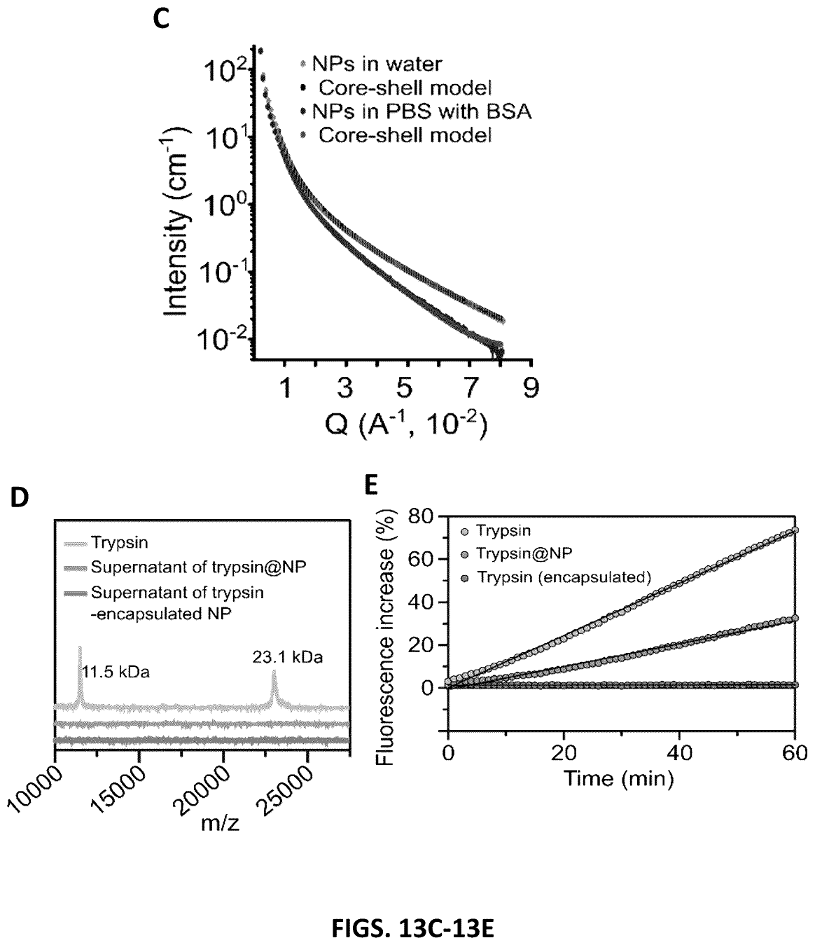

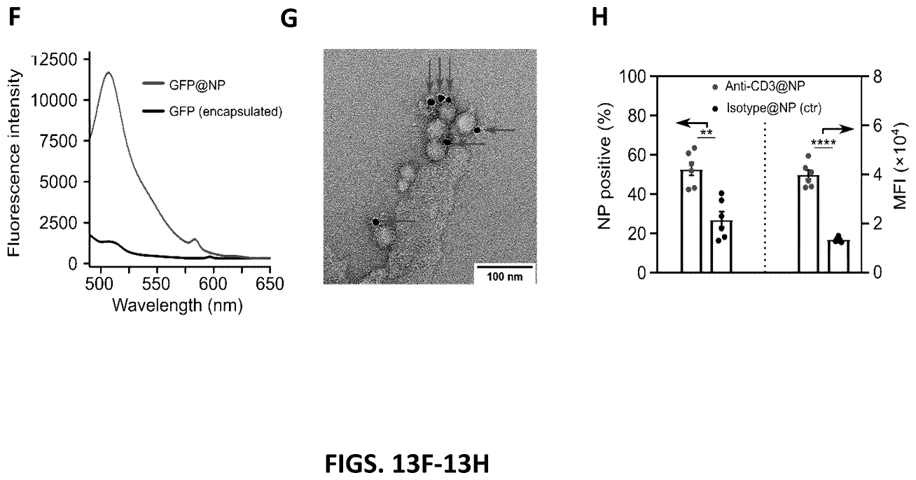

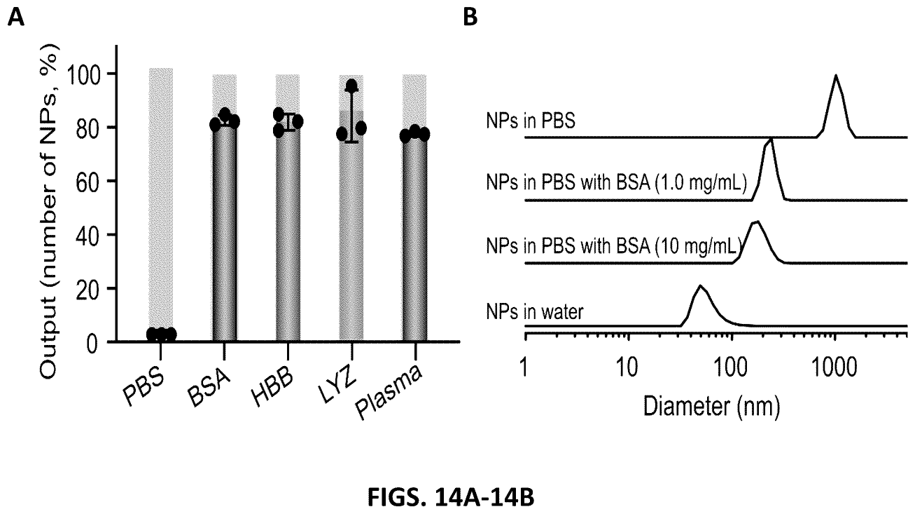

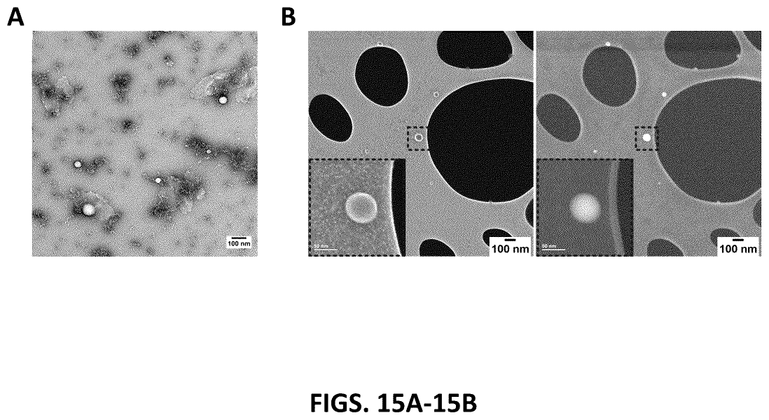

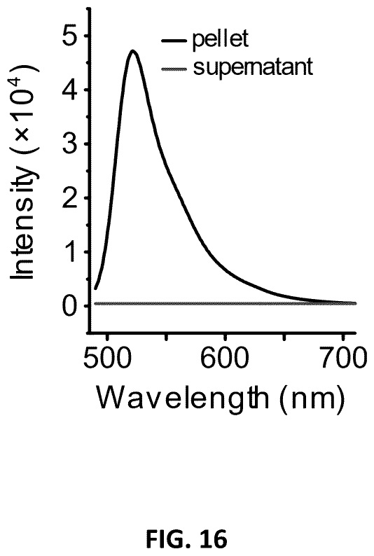

Having understood the distinct protein affinity of PPSU surfaces, we established a facile and versatile process for coating PPSU NPs with protein adlayers (FIG. 13A). In brief, PPSU NPs were incubated with excess proteins in PBS for 5 min at room temperature, followed by thorough washing to remove dynamically adsorbed proteins as well as residual proteins in solution. Saturation of adsorbed proteins on the NPs avoided aggregation in PBS (FIG. 14). The obtained protein-coated NPs showed improved colloidal stability, with their zeta potential dependent on the adsorbed proteins (FIG. 13B). Using BSA as a model protein, small-angle X-ray scattering (SAXS) measurements revealed that the shell thickness of NPs increased from 5.3 nm to 7.1 nm after protein coating (FIG. 13C). The BSA-coated NPs were imaged by transmission electron microscopy (TEM) and cryogenic scanning TEM (cryo-STEM), showing consistent sizes and morphologies with that of the pristine PPSU NPs (FIG. 15). To probe the displacement of adsorbed proteins by competitive serum proteins, we coated PPSU NPs with fluorescein isothiocyanate-tagged BSA (FITC-BSA) and incubated the complex of FITC-BSA@NP in pooled human plasma for 48 h at 37° C. FITC-BSA fluorescence was retained on pelleted NPs but was undetectable in the supernatant, confirming the irreversibility of the protein adsorption process (FIG. 16).

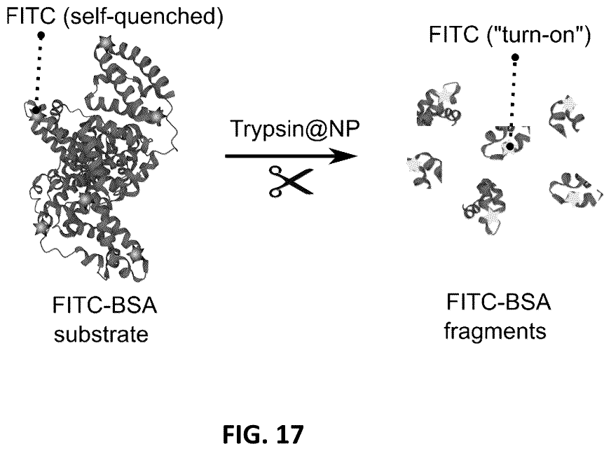

We explored the effect of irreversible adsorption on protein bioactivity using various adsorbed proteins as models. Trypsin and green fluorescent protein (GFP) were coated onto PPSU NPs to form complexes of trypsin@NP and GFP@NP, respectively. The stable surface presentation of adsorbed trypsin was demonstrated by matrix assisted laser desorption ionization time-of-flight (MALDI-TOF) mass spectrometry (FIG. 13D). To assess the enzyme activity of trypsin@NP, we quantified the significant increase in fluorescence resulting from the proteolytic cleavage of quenched FITC-BSA solutions into FITC-labeled peptides (FIG. 17). This assay confirmed the ability of trypsin@NP to cleave BSA (FIG. 13E). We previously reported that GFP had no detectable fluorescence after encapsulation within PPSU NPs, likely due to exposure to DMSO during NP formation⁸. In contrast, the structure-dependent fluorescence of GFP was readily detectable for GFP@NP (FIG. 13F), verifying surface adsorption to be a reliable and facile process for loading proteins without exposure to denaturing organic solvents.

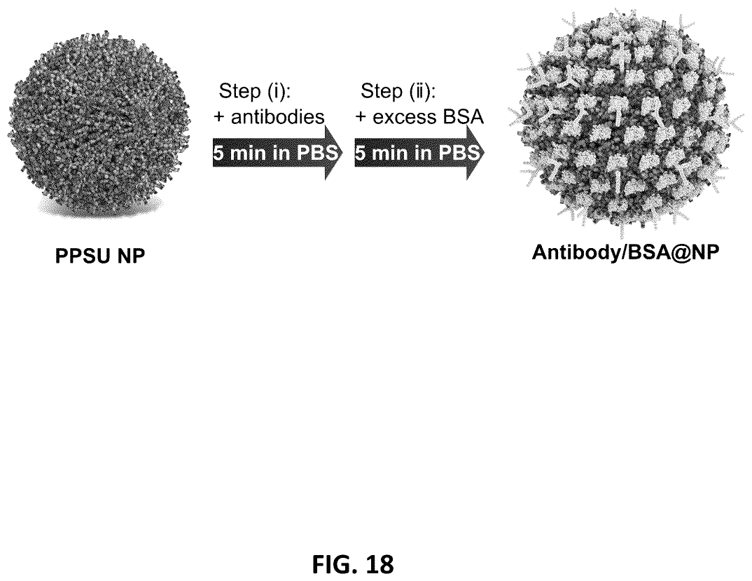

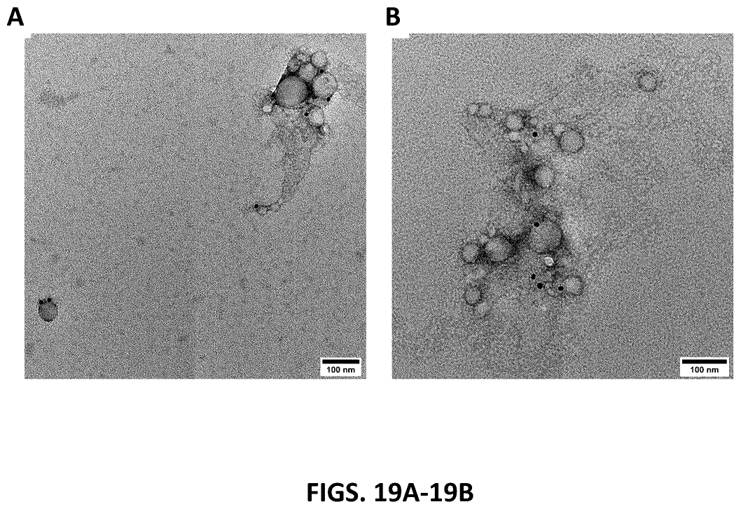

We next assessed the retention of antibody binding affinity, using low mass ratios of antibodies to achieve near 100% adsorption efficiency. Given the incomplete surface coverage of the antibody-adsorbed NPs, BSA as a blocking agent, was subsequently coated to avoid non-specific interactions with unoccupied regions on the NP surface. This two-step pre-adsorption protocol allows us to incorporate various and even multiple antibodies into protein adlayers with well-controlled composition (FIG. 18). Staining anti-CD4/BSA@NP with secondary gold-coupled antibodies verified the surface presence as well as immunological recognition by the pre-adsorbed primary antibodies (FIG. 13G and FIG. 19). Using CD3 as a cellular target, anti-CD3/BSA@NP significantly increased uptake into T cells compared to controls (FIG. 13H).

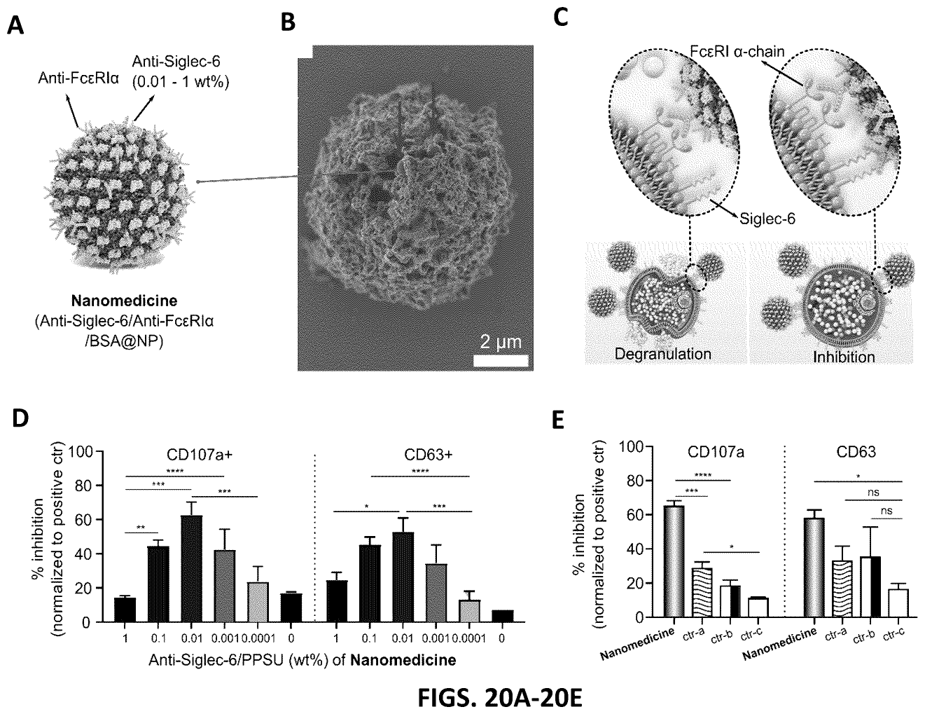

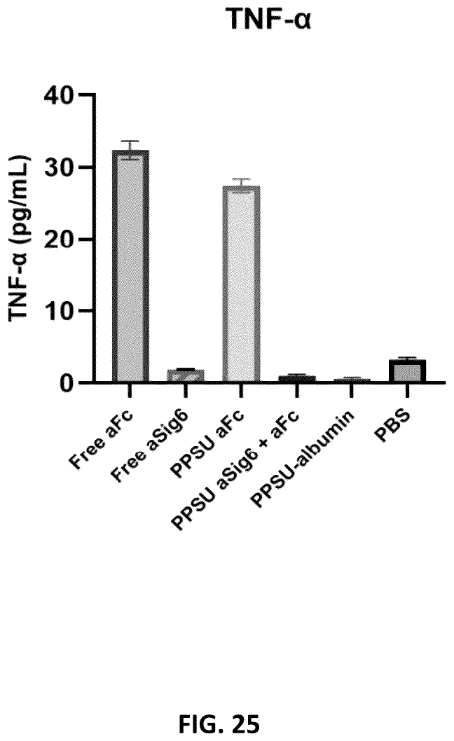

Mast cells (MCs) are tissue-based granulocytes involved in allergic and other responses and are historically difficult to selectively target with therapeutics²¹⁻²³. Perhaps the most significant barrier to developing selective therapies for MCs is the difficulty identifying specific surface targets to mediate inhibitory signaling upon FcεRIα activation following allergen binding. Siglec-6 has been identified as such a target^{10,11}, but optimizing inhibitory Siglec-6 signaling during FcεRIα engagement has not been achieved. We hypothesized that co-engagement of Siglec-6 with pre-adsorbed antibodies on PPSU NPs could be optimized to inhibit MC secretion for the prevention of anaphylaxis. To test this hypothesis, PPSU-based nanomedicines (FIG. 20A) that consist of co-adsorbed anti-Siglec-6 and anti-FcεRIα antibodies were prepared with a controlled surface density of anti-Siglec-6 antibodies. We confirmed the bioactivity of pre-adsorbed anti-FcεRIα antibodies to bind and activate primary human skin MCs in vitro, measured by TNF-α release (FIG. 25). The human skin mast cells were incubated with the test formulations of free antibodies and PPSU-bound antibodies for three hours. TNF-α was measured using a microfluidic ELISA kit. Secretion was best inhibited via co-localized engagement of Siglec-6 and FcεRI.

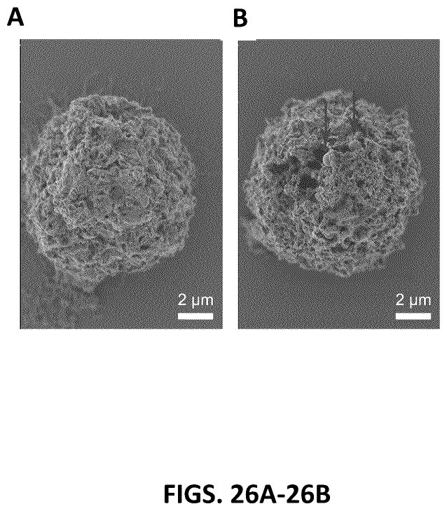

Importantly, inhibited secretion was observed when the nanomedicines were administered (FIG. 20B-20E and FIG. 26). This result suggested the ability of co-localized engagement to inhibit FcεRI-mediated activation of primary human mast skin cells (FIG. 20C).

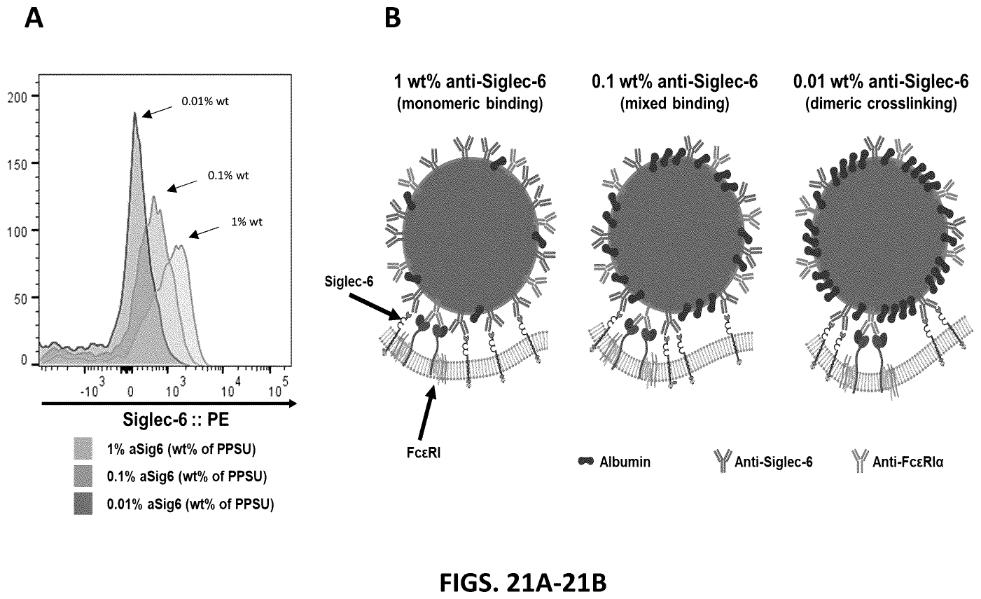

To further determine the optimal anti-Siglec-6 density for inhibition of degranulation, cells were stained for Siglec-6 after treatment with PPSU nanoparticles loaded with anti-Siglec-6 and anti-FcεRIα antibodies. As shown in FIG. 21A, flow cytometry results indicate decreased availability of Siglec-6 on the cell surface after treating cells with PPSU carrying decreasing densities of anti-Siglec-6, with the strongest decrease of Siglec-6 availability generated by an aSig6 density of 0.01 wt %. Therefore, lower densities of anti-Siglec-6 on PPSU bind more Siglec-6 receptor. Without being bound by any particular theory, it is believed that the increased PPSU binding to Siglec-6 receptor when loaded with lower densities of anti-Siglec-6 is due to an increase in dimeric crosslinking with the Siglec-6 on the cell surface, as illustrated in FIG. 21B.

MC degranulation cell surface markers (CD107a and CD63) were quantified via flow cytometry to assess the extent of mast cell activation. The experiments showed that lower densities of anti-Siglec-6 on NP surfaces resulted in greater receptor engagement and inhibition (FIG. 20D). The treatment was further evaluated and compared to a variety of controls, including a mixture of free form anti-FcεRIα with anti-Siglec-6/BSA@NP to assess the ability of a nanotherapy to inhibit pre- or co-activated MCs. The data indicated that localized co-engagement of both Siglec-6 with FcεRIα is necessary to achieve a significant reduction in degranulation (>60%; p<0.001), which was compared to control formulations where anti-FcεRIα was delivered without being co-adsorbed or without anti-Siglec-6 (FIG. 20E). Notably, these in vitro data demonstrated the need for both antibodies to be present on the NPs simultaneously.

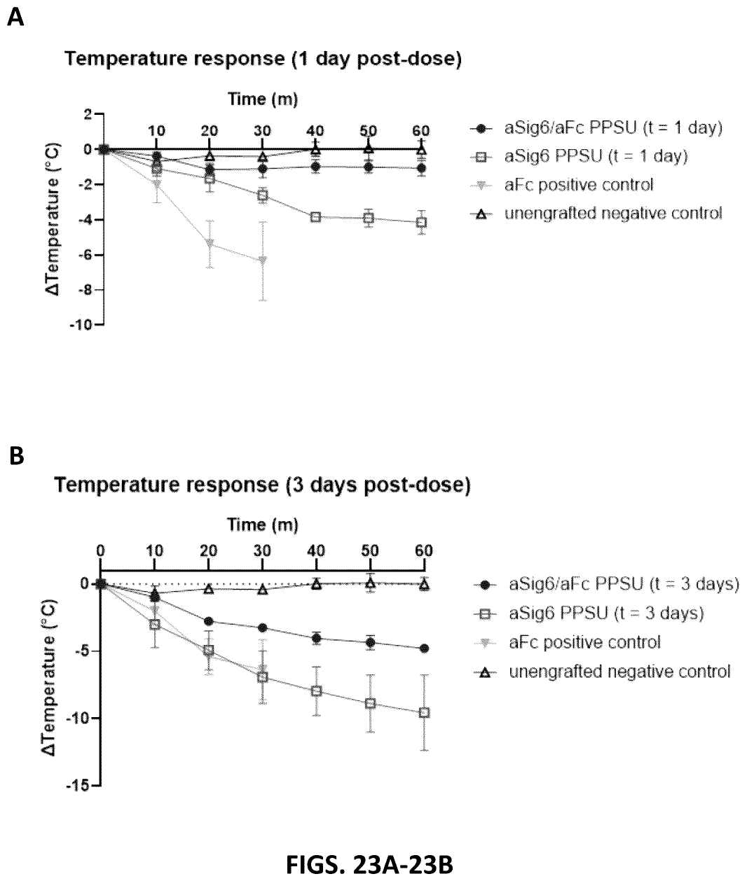

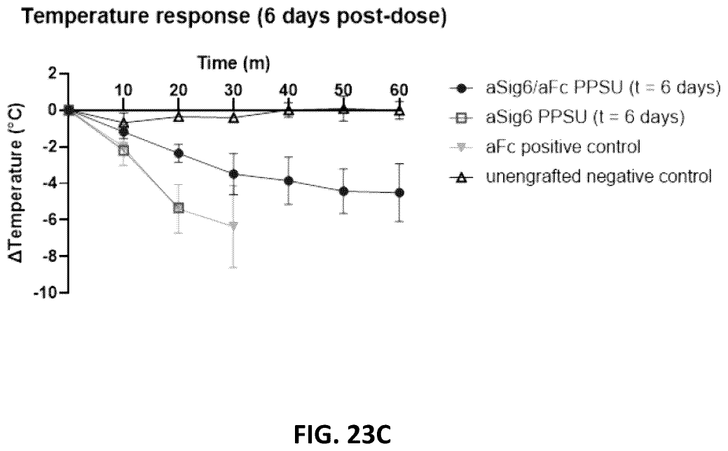

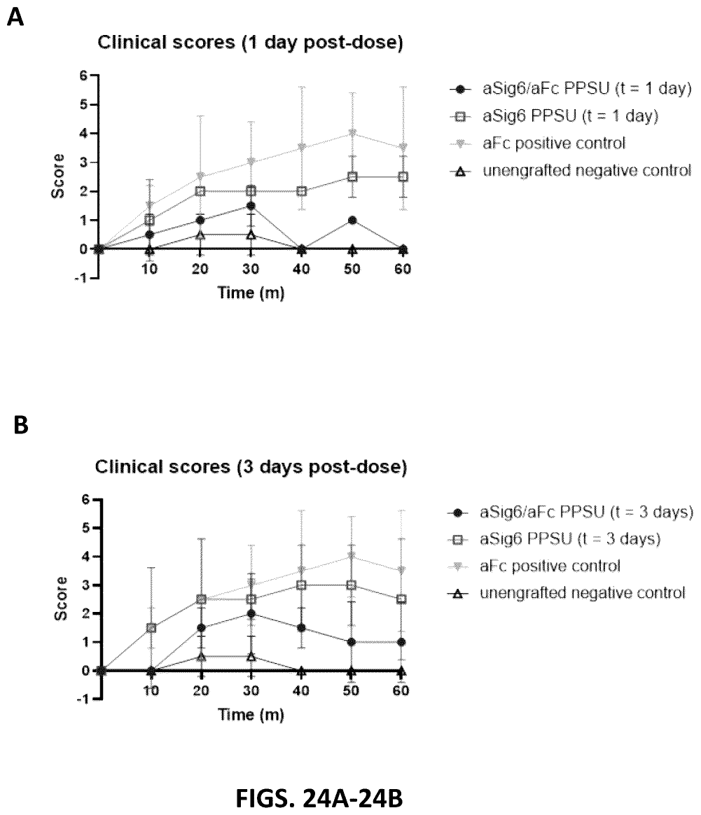

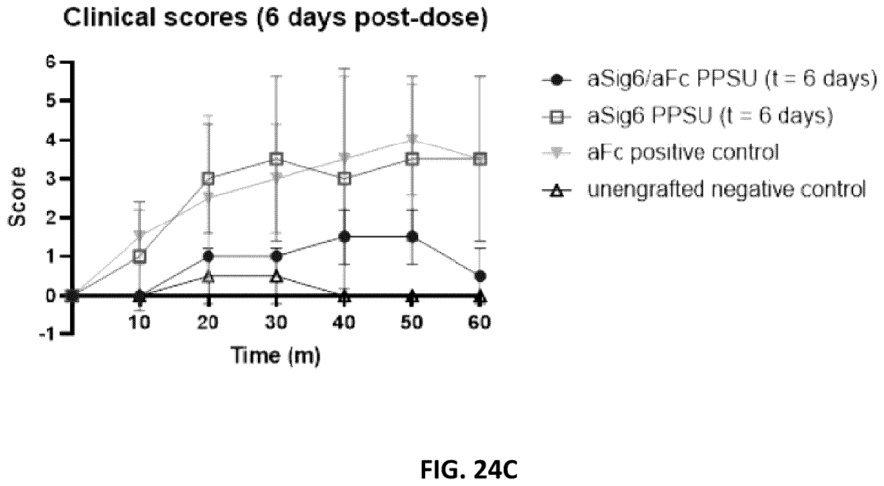

Similar experiments were performed to assess the duration of the protective effects of co-engagement of Siglec-6 and FcεRI. Mice were administered PPSU-bound anti-Siglec-6 and anti-FcεRIα at 6 days, 3 days, and 1 day before injection of free anti-FcεRIα antibodies. As shown in FIGS. 23A and 24A, after 1 day, aSig6/a Fc PPSU demonstrated a pronounced protective effect compared with particles that lacked both antibodies. However, mice that were dosed 3 days prior began to show decreased protection (FIGS. 23B and 24B). This decrease in protection remains at the same level for samples dosed 6 days prior (FIGS. 23C and 24C). In both cases, mice given aSig6 PPSU particles experienced more severe anaphylactic reactions, each consistent with mice given aFC alone. n=2 for all groups shown. In FIGS. 23A-C, groups that had mice die were cut off after that data point.

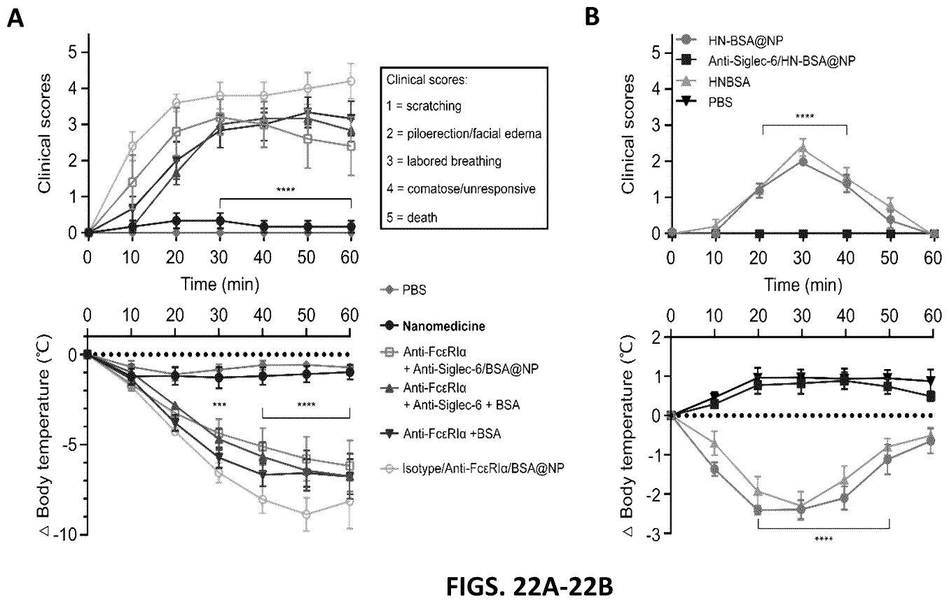

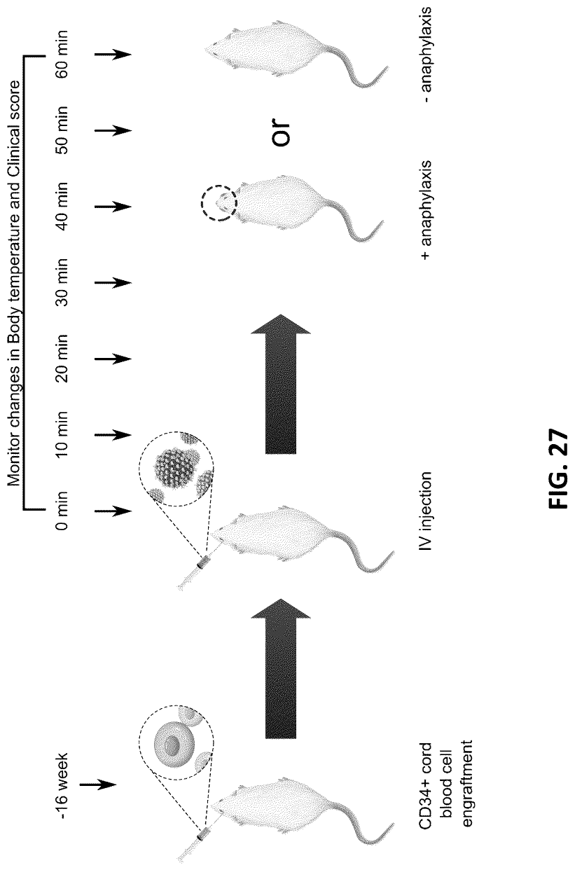

For in vivo validation, we tested the optimized nanomedicine formulation in a humanized MC mouse model by intravenous injection (FIG. 27). In this model, anaphylaxis was induced by utilizing anti-FcεRIα antibody to cross-link FcεRI and trigger mast cell degranulation. Anaphylactic reactions were measured by monitoring changes in body temperature using a digital rectal thermometer and a clinical scoring system (FIG. 20F)²⁴. Mice that experienced anaphylaxis had significantly greater drops in body temperature and increased clinical scores (ΔT>5° C., clinical scores >2, p<0.001)). Successful inhibition of anaphylaxis was manifested by responses that were statistically indistinguishable to the PBS negative control (ΔT≤1° C., clinical score <0.5). Mice treated with the nanomedicine displayed near-complete inhibition of anaphylaxis, whereas other formulations which did not have antibodies co-adsorbed or were freely solubilized did not display any significant inhibition of anaphylaxis. These findings confirmed our in vitro data, which suggested that co-engagement is necessary for optimal inhibition of anaphylaxis (FIG. 20E). This is likely due to spatial-temporal requirements of Siglec-6 signaling in proximity to FcεRI during activation. We and other groups have demonstrated that engaging Siglecs and other ITIM-containing receptors (Siglec-6 included) recruits the phosphatases SHP-1/SHP-2 and SHIP, which in turn dephosphorylate and disrupt tyrosine kinase activity resulting from FcεRI signaling²⁵⁻²⁷. While it is possible to achieve co-engagement by flooding in vitro assays with both antibodies in soluble form to achieve inhibition, it is much more difficult to achieve in vivo without using large amounts of antibodies to ensure that both receptors are engaged at the same time. By utilizing PPSU NPs as a platform to unite both antibodies in proximity, we can maximize co-localization of both signals and achieve what we see in FIG. 22A.

We also tested a model for inhibiting IgE-mediated allergy, in this case we utilized 4-Hydroxy-3-nitrophenylacetyl hapten conjugated to BSA (HN-BSA) to simulate allergic sensitivity to a hapten molecule^{24,28}. The resulting data (FIG. 22B) mirrored the findings of the above experiment which utilized anti-FcεRIα; PPSU vesicles coated with anti-Siglec-6 and HN-BSA demonstrated significant inhibition (p<0.0001, n=5) when compared to HN-BSA@NP and solubilized HN-BSA when tested in vivo. Inhibited mice and PBS control mice demonstrated a small rise in body temperature after initial measurements at t=0 minutes. This was likely an artifact of the cooler ambient temperature of a new testing room (˜19° C.), the mice rousing from a resting state. Body temperatures rose from around 36.5 to 37.5° C. In contrast, our HN-BSA@NP and unbound HN-BSA groups demonstrated drops in body temperatures averaging around ⁻2.5° C. This is consistent with past experiments which have observed drops ranging from mild (⁻1.5° C.) to severe or fatal (⁻10° C.)^{24,29-31}.

The change in body temperature observed in these experiments was not as extreme as seen in our anti-FcεRIα antibody model, which saw drops between −6 and −9° C. Two key differences in this model were the significantly different material sensitivities for anaphylaxis (100 μg HN-BSA vs 100 ng anti-FcεRIα) and the need to sensitize mice 18-24 hrs in advance with IgE. Together these factors presented entirely different challenges to effectively triggering anaphylaxis in vivo. We selected HN-BSA as our allergen due to it being a hapten²⁸. Haptens are small molecules bound to larger molecules such as serum proteins, that when combined, act as antigens capable of cross-linking FcεRI-bound IgE. They are advantageous in allergy models due to only requiring one form of IgE to bind the hapten epitope. In comparison, most anaphylactic reactions require multiple forms of IgE to bind different epitopes of the allergen to achieve FcεRI cross-linking³². While the PPSU vesicle platform is likely capable of supporting this approach, it would require extensive optimization to study in vivo. Another advantage of the anti-FcεRIα and anti-Siglec-6 PPSU vesicle is that it is not necessarily limited to a specific allergy, and therefore has the potential to broadly inhibit mast cells. If allergen-specific therapies were developed, it would likely require a separate regulatory process for each one—which would be cost-prohibitive and inefficient. These facts, combined with the sensitivity and potency of the anti-FcεRIα antibody-mediated mouse model, appear to indicate a greater feasibility of antibody-based methods as a therapeutic when compared to allergen-based approaches.

Using a combination of in silico, in vitro and in vivo methods and a simple mixing protocol, we demonstrate controlled, irreversible adsorption of multiple proteins simultaneously to PPSU NP surfaces while preserving protein function. Highly dynamic PPSU surfaces were capable of site-to-site hydrophilic/hydrophobic switching in the presence of adsorbing protein, which allowed facile binding of bioactive enzyme and antibody combinations. This design freedom provided the flexibility to optimize a novel nanotherapy for anaphylaxis, achieving the first targeted inhibition of FcεRI receptor activation on human mast cells via Siglec-6 signaling. Our work validates PPSU as a robust platform for the facile engineering of bioactive NPs and allows customization of the complex and combinatorial capabilities of biologics in the treatment of disease.

Methods for FIGS. 2, 7, 13, 20-24

Preparation of PPSU NPs. PPSU was prepared in our previous report⁸. Self-assembly of PPSU was performed via stepwise hydration using the confined impingement jets mixer. Typically, 15 mL of PPSU solution (25 mg/mL in DMSO) and 15 mL of water were impinged against one another within the CIJ mixer by syringe injection. This process was repeated once more using 15 mL of water and the obtained 30 mL of suspension as the two impingement solutions. After the two-step hydration, 45 mL of suspension was collected, followed by dialysis in water (3 days) to remove DMSO. As prepared, the NPs (suspended in water) were stored at room temperature before using.

Cryo-scanning transmission electron microscopy (cryo-STEM). Lacey carbon grids (200 mesh, Electron Microscopy Sciences, Inc.) were glow-discharged for 20 s in a Pelco easiGlow glow-discharger (15 mA, 0.24 mBar). Each sample (4 uL) was pipetted onto a grid and plunge-frozen into liquid ethane using an FEI Vitrobot Mark IV cryo plunge freezing robot with a blotting time of 5 or 5.5 seconds and blotting pressure of 1. Frozen grids were stored in liquid nitrogen and then loaded into a Gatan 626.5 cryo transfer holder cooled down to −180° C. prior to image within a Hitachi HD2300 STEM at 200 kV. Frozen samples were previewed with the phase contrast TE and HAADF detectors to verify sample preparation. With the sample still in the microscope, the cryo holder was slowly warmed up to −95° C. over the course of 30 minutes to sublime away ice within the vacuum. Imaging in the microscope continued using an SE detector in the STEM to image the surface of our particles. Image data was collected with Gatan Digital Micrograph and a Digiscan system. Any further image processing conducted on the aligned frames was completed in ImageJ.

All atom explicit solvent molecular dynamics simulations. All simulations were performed with GROMACS 2021.5³³, an MD package mainly designed for simulations of large biomolecules. The CHARMM 36 m force field 34 was used for the all-atom simulations, which has been validated in our last work⁸. The recommended CHARMM TIP3P water model was applied with the structures constrained using the SETTLE algorithm³⁵. All visual analyses were performed with VMD³⁶. In all the simulations, the degree of polymerization of 20 was employed for the polymer chains, the same as that PPSU₂₀ in the experiments.

Preparation of protein@NP complex via protein adsorption. Except for antibodies, excess protein was used to achieve full cover on NP surfaces. (i) NPs (5 mg/mL) were suspended in protein solution (10 mg/mL in PBS) for 5 min; (ii) they were centrifuged, and the NP pellet was collected; (iii) the pellet was resuspended in PBS, followed by centrifugation. Step (iii) was repeated a total of three times to remove unbound proteins. The final protein@NP complex was suspended in PBS.

Encapsulation experiments. Rhodamine B was encapsulated during the formation of PPSU NPs. 200 μL of PPSU solution (25 mg/mL in DMSO) was thoroughly mixed with 200 μL of aqueous solution of rhodamine B (0.01 mg/mL), then 200 μL of water was added and mixed thoroughly. The mixture (600 μL) was dialyzed in water for 3 days to completely remove DMSO and free rhodamine B. The encapsulation efficiency of rhodamine B is nearly 100% by fluorescence measurements. Trypsin was encapsulated using an overloading method. 200 μL of PPSU solution (25 mg/mL in DMSO) was thoroughly mixed with 200 μL of aqueous solution of trypsin (20 mg/mL), then another 200 μL of the trypsin aqueous solution was added and mixed thoroughly. After the addition of 1.4 mL of water, the mixture (2 mL) was centrifuged, and the pellet was resuspended in water. Repeating the water washing steps for a total of three times. All the supernatant was collected to quantify the free trypsin using UV spectroscopic analysis technique, showing a saturation encapsulation/adsorption of ˜0.96 mg of trypsin per 1 mg of NPs.

Trypsin proteolysis fluorescence-based kinetic assay. 2 ug/ml of trypsin (free protease or nanoparticulate form) was incubated with 1 mg/mL of quenched FITC-BSA substrate at 37° C. Trypsin cleaves yields FITC-labeled peptides derived from the full-length protein, which releases the fluorophore from a quenched state and results in an increase in fluorescence. Proteolysis was therefore monitored by measuring the increase in fluorescence over a 1.5 h period. (1 measurement per minute). Measurements were obtained in three replicates per condition.

SAXS measurements. SAXS measurements were performed at the DuPont-Northwestern-Dow Collaborative Access Team (DND-CAT) beamline at Argonne National Laboratory's Advanced Photon Source (Argonne, IL, USA) with 10 keV (wavelength λ=1.24 Å) collimated X-rays. All the samples (5 mg/mL) were analyzed in the q-range (0.001-0.5 Å⁻¹), with a sample-to-detector distance of approximately 8.5 m and an exposure time of 5 s. The beamline was calibrated using silver behenate and gold coated silicon grating with 7,200 lines/mm The momentum transfer vector q is defined as q=4π sin θλ⁻¹, where 20 is the scattering angle. Data reduction and buffer subtraction were performed using IRENA 2.71 package within IGOR PRO 9 software (Wavemetrics). Model fitting was completed using SasView 5.0.5 software package where core-shell sphere model was utilized to fit and analyze the data.

MALDI-TOF mass spectrometry detection of trypsin. Trypsin (5 wt %) was encapsulated or adsorbed by PPSU NPs. As prepared, the samples were stored for 48 h at 4° C. to permit leakage of weakly associated trypsin (if present). The samples were pelleted by centrifugation at 20,000×g for 10 min at room temperature. The supernatants were collected and were mixed 1:1 with sinapinic acid matrix prepared in ultrapure water with 0.2% trifluoroacetic acid. Trypsin prepared at 2 ug/mL was used as a positive control for detecting full length protein. The supernatant collected from PPSU NPs prepared in the absence of protein was used as a negative control. These mixtures were spotted on a stainless steel MALDI disc (Bruker), and full-length trypsin protein (˜23 kDa) was detected by MALDI-TOF mass spectrometry. Positive ion mass spectra were collected on a Bruker rapifleX MALDI Tissuetyper operating in linear TOF mode. Spectra were obtained in triplicate.

Immunogold labelling. Samples were prepared by mixing 90 μL of PPSU NPs (1 mg/mL in water), 10 μL of anti-CD4 antibodies (0.5 or 0 mg/mL in PBS), 10 μL of 10×PBS, and 100 μL of BSA (1 mg/mL in PBS). After incubation for 5 min at 4° C., the samples were centrifuged, and the collected pellets were resuspended in PBS. This step was repeated a total of three times to remove unbound protein molecules. 20 μL of secondary gold-coupled antibodies (with 1 wt % of BSA in PBS) was added into each of the samples and the mixtures were dialyzed in water to remove salts. Negatively stained grids were prepared using 0.5 mg/mL of NPs in water, then visualized on a JOEL 1400 TEM operating at 120 kV.

Cellular uptake studies. Rhodamine B-encapsulated NPs were mixed with 5 wt % of anti-CD3 antibodies (or Isotype control, 10 ug/mL) in PBS for 5 min, then suspended in PBS with 10 mg/mL of BSA. Unbound proteins and any leaked rhodamine B were removed by washing with PBS. The antibody-adsorbed NPs (25 μg/mL) were incubated with Jurkat T cells for 30 min at 37° C. Cells were analyzed using a BD Fortessa flow cytometer. The cellular uptake was measured as % of NP positive cells and median fluorescence intensity (MFT).

Preparation of formulations for anti-FcεRIα-mediated in vitro and in vivo validation experiments. Stocks of antibody-adsorbed NPs were prepared by concurrently incubating PPSU NPs with set concentrations of anti-FcεRIα (0.1 wt % for in vitro or 0.2 wt % for in vivo) and variable amounts of anti-Siglec-6 to create NPs for co-engagement. Anti-Siglec-6 concentrations for in vitro experiments were determined by loading different weight percentages of the total PPSU mass present in each sample (0.01, 0.1, and 1 wt %). This translated to final well concentrations of 8 ng/mL, 80 ng/mL, and 800 ng/mL, respectively. In mouse experiments, we utilized 2.5 wt % anti-Siglec-6 (511 g per injection). When preparing PPSU NPs with multiple antibodies, the protein was added to a microtube in the desired ratios and concentrations before addition of PPSU NPs. After incubation of antibodies for 5 min, NPs were incubated with excess BSA before final washes to remove unbound protein. The NPs and antibodies were incubated at room temperature in PBS.

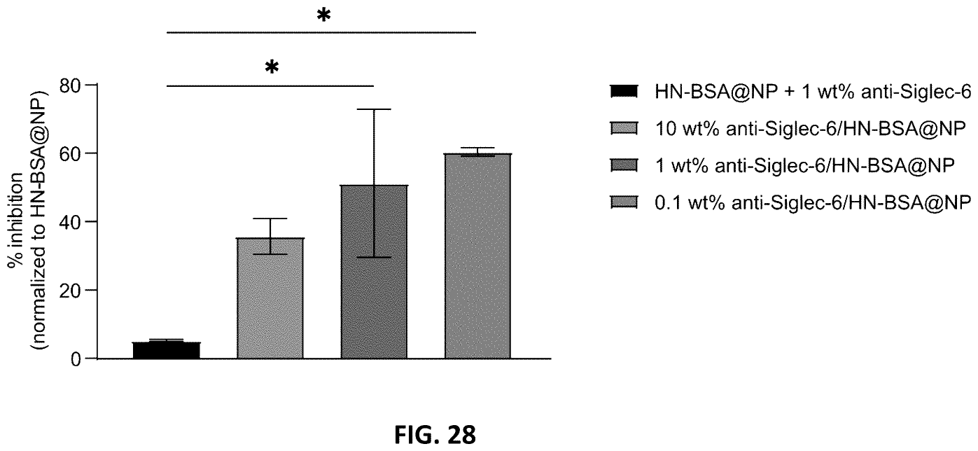

Preparation of formulations for IgE-mediated in vitro and in vivo validation experiments. Stocks of PPSU NPs for in vivo experiments composed of anti-Siglec-6 antibody and/or 4-Hydroxy-3-nitrophenylacetyl hapten conjugated to BSA (HN-BSA) were prepared by sequentially incubating PPSU NPs (100 μg) with set concentrations of (1) anti-Siglec-6 (10 wt %) for 10 minutes followed by (2) HN-BSA (200 μg). Prior to injection, HN-BSA was quantified by measuring samples on a UV-Vis spectrometer at 430 nm and adjusting to a final dose of 100 μg per mouse. In vitro samples (FIG. 28) were prepared in the same manner, with different concentrations of reagents to better fit cell culture conditions. For this, we incubated PPSU NPs with 0.25 wt % of HN-BSA and a range of anti-Siglec-6 concentrations (10 wt %, 1 wt %, and 0.1 wt %). Samples were formulated to achieve a final concentration of 100 ng/mL HN-BSA. All in vivo and in vitro samples were prepared, washed, and suspended in 1×PBS prior to use.

Mast cell isolation and cell culture. Skin-derived primary mast cells (SkMCs) were isolated from 100 cm² human skin samples received from the Midwestern Division of the Cooperative Human Tissue Network. Multiple donors were used across our experiments to ensure consistency despite patient variability. We utilized a protocol developed by Caslin et al.³⁷; in brief, we cut skin into <1 cm² pieces and then subjected tissue to a digestive enzyme wash at 37° C. for 1 hour, completing 3 rounds of washes. After this, cells were pelleted under centrifugation and further purified by collecting a buffy coat over percoll. Cells were then purified (>95%) via passive media selection in culture for 8 weeks before use in experiments. The protocol is outlined in FIG. 1E.

Scanning electron microscopy (SEM). The medium was removed after cell incubation, and glutaraldehyde solution (2.5%) was added for fixation overnight at 4° C. The cells were then rinsed three times with 0.1M of sodium cacodylate buffer and once with water (5 min) and incubated in osmium tetroxide (2%) for 1 hour. After staining, the cells were rinsed another three times with water (10 min per time). The cells were dehydrated with a series of ethanol gradients (30, 50, 70, 85, and 95%) for 10 min at each step. The dehydrated cells were rinsed with 100% ethanol two times (10 min per time) and was completely dried using Samdri®-795—tousimis Critical Point Dryers. After drying, the cells were placed on a carbon tape attached to an aluminum stud and coated with SPF Osmium Coater (10 nm thickness) for SEM analysis. The morphology was then observed by a Hitachi SU8030 scanning electron microscope.

Analysis of degranulation markers with flow cytometry. In preparation for the experiments, we used dose curves to optimize the concentration of anti-FcεRIα required to induce clear expression of degranulation markers on SkMCs above baseline levels (80 ng/mL). Additionally, we incubated the cells with 10 ng/mL of polyclonal human IgE (Abcam) 0/N to stimulate FcεRI expression and prime cells for activation. For the experiment, SkMCs were resuspended in ice cold Tyrode's buffer at 300K cells/sample and given a variety of experimental formulations containing PPSU NPs, free or antibody-adsorbed NPs, or combinations of each. Mouse IgG2a isotype antibody (BioLegend clone MG2a-53) was used as a control comparison for anti-Siglec-6. Cells were placed in a 37° C. incubator for 20 minutes before being placed immediately back on ice to halt ongoing degranulation. Cells were then washed with PBS and suspended with live/dead fixable blue dead cell stain for 30 minutes at 4° C. Subsequently, cells were washed with FACS buffer (PBS with 3-4% FBS) and resuspended with blocking buffer for 10 minutes and then directly added master mix of antibody labels to incubate for 45 minutes at 4° C. At last, cells were washed 2 times with FACS buffer or fixed with 4% PFA and then washed, depending on equipment availability. All flow sample data were collected on a BD FACSymphony and simultaneously compensated using the equipped X software. Antibodies: BV421 labeled anti-CD117 (KIT), clone 104D2 from BioLegend. PE labeled anti-Siglec-6, clone 767329 from R&D Systems, PE/Cy7 labeled anti-CD63, clone H5C6, from BioLegend, APC labeled anti-CD107a (lamp 1), clone H4A3, from BioLegend, and APC/Cy7 labeled anti-FcεRIα, clone AER-37, from BioLegend. Samples were analyzed on FlowJo v10. All samples were gated for singlets, then live/dead and normal KIT expression before analyzing other markers. Additionally, all samples were gated to a minimum of 15,000 KIT+ cells.

Cytokine expression analysis via enzyme-linked immunosorbent assay (ELISA). Human TNF-α (cat. #QTA00C) ELISA kits were obtained from R&D systems. 100K SkMCs cells/well (n=4 per test group) were incubated with formulations for 3 hours. The supernatant was then collected and stored overnight at −20° C. before being used in kits as prescribed in their protocols. The same conditions were utilized for our other in vitro experiments. Here, we dosed cells with 0.1 wt % anti-Siglec-6 (80 ng/mL) on PPSU NPs, with a final well concentration of 80 μg/mL of PPSU NPs (8 μg per sample). Just as with the flow cytometry samples, anti-FcεRIα was adsorbed onto PPSU NPs or dosed separately at 80 ng/mL.

Humanized mouse engraftment and anaphylaxis model. We utilized a previously established and characterized model to test the inhibitory effects of our formulations in vivo²⁴. In step 1, PPSU homopolymer was dissolved in DMSO. In step 2, water was added to the solution in 50 μL increments to allow aggregation of the homopolymers into PPSU nanoparticles. In step 3, dialysis was performed to remove residual DMSO. In step 4, desired proteins, e.g. anti-FcεRIα antibodies and anti-Siglec-6 antibodies, were added to the solution. Albumin was also added as a capping/filler agent. The samples were then purified by centrifugation. Four-week-old NSG-SGM3 mice were retro-orbitally injected with hCD34+ stem cells. After 16 weeks, mice were checked for engraftment using flow cytometry. We used FITC labeled anti-hCD45 clone 2D1 (BioLegend) and APC/Cy7 labeled anti-mCD45 clone 103116 (BioLegend) to compare human and mouse cell populations. Successfully engrafted mice (>5% hCD45+) were then randomized with respect to the percentage of engraftment and retro-orbitally injected with test formulations. Nanoparticulate and free anti-FcεRIα (1 μg/mL) was used as a positive control to comparison to mice given the nanomedicine and the other test formulations. Visual clinical scoring and a rectal temperature probe were used to track anaphylactic reaction severity for 1-hour post-injection, with data collected at 10-minute intervals. Clinical scores were determined as follows: 0: asymptomatic 1: scratching, 2: piloerection/facial edema, 3: labored breathing, 4: comatose/unresponsive 5: death. Mice were then sacrificed and not reused. The protocol is outlined in FIGS. 1C-1D and 27.

Humanized anaphylaxis model of IgE-mediated allergy. 13-to-18-week-old NSG-SGM3 mice pre-engrafted with hCD34+ stem cells were acquired from The Jackson Laboratory. This experiment directly adapts previous methods which utilized engrafted NSG-SGM3 NSG-SGM3 mice^{24,29-31}. We purchased 4-hydroxy-3-nitrophenylacetyl hapten conjugated to bovine serum albumin (HN-BSA, Biosearch Technologies) with a ratio of 8 HN units per BSA protein. We also purchased anti-Siglec-6 antibody (aSig6, R&D Systems: clone 767329). Hybridoma-generated anti-HN chimeric human IgE was donated by Allakos, Inc. Each mouse was given a single retro-orbital intravenous injection of 1.6 μg IgE in 100 μL of PBS to sensitize them to HN hapten. The following day (18-24 hrs later), mice were injected with experimental or control reagents in 100 μL of PBS. Reagents were formulated as previously described; in brief: PPSU NPs (100 μg) were incubated with anti-Siglec-6 (10 μg) and an excess of HN-BSA (200 μg) or PPSU NPs were incubated with HN-BSA (no anti-Siglec-6) using the same amounts. Anti-Siglec-6 was mixed with NPs 20 minutes before HN-BSA to reduce competition and ensure consistent adsorption of the antibody. We utilized 100 μg of solubilized HN-BSA in PBS as a positive control, and a PBS vehicle control. In particle samples, HN-BSA concentrations were measured on a UV-Vis spectrometer at the absorption peak of HN (430 nm) and concentrations were adjusted to a final dose of 100 μg of HN-BSA in 100 μL per mouse. Mouse temperatures and clinical scores were measured as previously noted in the anti-FcεRIα anaphylaxis model.

Methods for FIGS. 3-6, 8-12, 14-19, 25-28

Chemicals and materials. All chemical reagents were purchased from Sigma-Aldrich and were used without further purification unless otherwise indicated. Antibodies were acquired from BioLegend. Pooled human plasma was used after nanofiltration (100 nm). Recombinant A. victoria GFP protein (from the Jewett Lab). Goat Anti-Mouse IgG (10 nm Gold, Abcam) was used for immunogold labelling.

Dynamic light scattering (DLS). Size distribution and zeta potential of NPs were analyzed by Zetasizer Nano (Malvern Instruments) with a 4 mW He—Ne 633 nm laser at 22° C. The polydispersity index (PDI) was calculated using a two-parameter fit to the DLS correlation data.

Transmission electron microscopy (TEM). Formvar carbon film copper grids (400 mesh, Electron Microscopy Sciences, Inc.) were used immediately after glow-discharging (25 W, 20 s). Samples were applied to the grids, followed by negative staining (1.5% uranyl formate, two times). Excess stained was removed by blotting each sample with Whatman filter paper. TEM images were acquired on a JOEL 1400 Transmission Electron Microscope operating at 120 kV. Any further image processing conducted on the aligned frames was completed in ImageJ.

Cryo-transmission electron microscopy (cryo-TEM). Samples were prepared by applying 3 μL of suspension on pretreated holey lacey carbon 400 mesh TEM copper grids (Electron Microscopy Sciences). Following a blot of 3 s, samples were plunge-frozen (Gatan Cryoplunge 3 freezer). Images of samples entrapped in vitreous ice were acquired using a field emission transmission electron microscope (JEOL 3200FS) operating at 300 keV. Digital Micrograph software (Gatan) was used to align the individual frames of each micrograph to compensate for stage and beam-induced drift. Any further image processing conducted on the aligned frames was completed in ImageJ.

Fluorescence and UV-Visible spectroscopy. Fluorescence spectra were recorded by RF-6000 using LabSolutions RF software. UV-Visible absorbance and transmittance were measured by Perkin Elmer LAMBDA 1050.

Protein quantification. The concentrations of proteins were quantified by UV-Visible absorbance or fluorescence depending on protein type and equipment availability. NanoDrop Abs was used for testing trace samples.

All atom explicit solvent molecular dynamics simulations. The analysis of the 6-chain PPSU₂₀ aggregate system was based on the simulations reported in our previous work¹. For the 125-chain NP system, 125 PPSU₂₀ chains were distributed spherically with a vacancy in the center of the NP using PACKMOL². The NP has an initial outer radius of 7.5 nm and an initial vacancy radius of 2.5 nm. For the 600-chain NP system, 600 PPSU₂₀ chains were distributed spherically with a vacancy in the center of the NP using PACKMOL. The NP has an initial outer radius of 8 nm and an initial vacancy radius of 4.5 nm. Each NP is dissolved in a water solvent box with a dimension of 20×20×20 nm³. For each NP-water system, the potential energy was first minimized using the steepest descent algorithm for 1,000 steps with a time step of 1 fs. A subsequent NVT ensemble (constant number of particles, volume, and temperature) equilibration of 1 ps was performed. Subsequently, the NPT ensemble (constant number of particles, pressure, and temperature) was applied for equilibration of 1 ns using the time step of 2 fs. Subsequently, long MD production simulations were performed. The periodic boundary conditions were applied in all three dimensions. The neighbor searching was performed up to a cut-off distance of 1.2 nm utilizing the Verlet particle-based approach and was updated every 20 timesteps. The potential-switch method was applied for the short-range Lennard-Jones (LJ) 12-6 interactions from 1 nm to 1.2 nm. The short-range electrostatic interactions were calculated up to 1.2 nm, and the long-range electrostatic interactions were calculated employing the Particle Mesh Ewald algorithm³. A time step of 2.5 fs was employed by constraining all the covalent bonds using the LINCS algorithm⁴. The temperatures of the PPSU₂₀ solute and the solvent molecules were separately coupled using the Nosé-Hoover algorithm s (reference temperature 298 K, characteristic time 1 ps). The isotropic Parrinello-Rahman barostat 6 was utilized with the reference pressure of 1 bar, the characteristic time of 4 ps, and the compressibility of 4.5×10⁻⁵ bar⁻¹. Each of the simulations run 200 ns. The final simulation snapshots are presented in FIG. 2B. The interaction energies were calculated with gmx energy integrated within the GROMACS 2021.5 package.

The structure of trypsin was downloaded from Protein Data Bank (www.rcsb.com) with the PDB ID of 1S0Q. After the simulation of 600-chain NP approached equilibrium, the simulation box was enlarged to 22×22×22 nm³. Subsequently, 6 trypsin molecules were solvated in the system, along with counter ions (60 Cl⁻) to balance the charge of the 6 trypsin molecules. The trypsin molecules were distributed randomly and close to the NP surface. Energy minimization, NVT equilibration, NPT equilibration, and 200 ns MD production run were performed with the same simulation parameters aforementioned. Subsequently, the number of —SO₂— groups on the NP surface was counted. In this work, the —SO₂— groups that are within 2.5 Å of the water solvent molecules or protein molecules are considered as surface groups. The number of hydrogen bonds formed between trypsin and water was calculated with a criteria of θ_{HDA}≤30° and r_{DA}≤3.5 Å between the hydrogen (H), donor (D), and acceptor (A)⁷. The binding energy between trypsin molecules and the NP was calculated with the gmx energy tool. The compositions of the trypsin binding domains were analyzed with our recently developed program Protein Surface Printer⁸. The results indicated there were no preference for binding orientations of trypsin.

The protein structure of Bovine Serum Albumin (BSA) was downloaded from Protein Data Bank with the PDB ID of 4F5 S. After the simulation of 600-chain NP approached equilibrium, the simulation box was enlarged to 20×19×24 nm³. Subsequently, 2 molecules of BSA were solvated in the system, along with counter-ions (32 Na t) to neutralize the net charge of the system. The BSA molecules were distributed randomly in the simulation box. Energy minimization, NVT equilibration, NPT equilibration, and 200 ns MD production run were performed with the same simulation parameters aforementioned. During the simulation, one of the BSA molecules diffused slowly in solvent and was adsorbed onto the NP near the end of the simulation. Therefore, quantitative analysis was only carried out on the BSA that stably bound to the NP. The number of surface —SO₂— groups of the NP was counted in the similar manner as that in the NP-trypsin system. The number of hydrogen bonds formed between BSA and water was calculated using the same structural criteria as those in the NP-trypsin system. The binding energy between BSA and the NP was calculated with the gmx energy tool.

The structure of IgG1 human antibody was downloaded from Protein Data Bank with the PDB ID of 1IGY. For IgG1-PPSU complex system, 3 parallel simulation runs were performed. After the simulation of 600-chain NP approached equilibrium, the simulation box was enlarged to 30×30×30 nm³. Subsequently, 1 molecule of IgG was solvated in the system, along with counter-ions (20 Cl⁻) to neutralize the net charge of the system. The IgG1 molecules were distributed such that in the first simulation, the Fc domain was facing the PPSU NP, in the second simulation, one of the Fab domains was facing PPSU NP, and in the last simulation, the IgG1 molecule lay flat on the PPSU NP surface. Energy minimization, NVT equilibration, NPT equilibration, and 200 ns MD production run were performed with the same simulation parameters aforementioned. The number of surface —SO₂— groups of the NP was counted in the similar manner as that in the NP-trypsin system. The number of hydrogen bonds formed between IgG1 molecule and water was calculated using the same structural criteria as those in the NP-trypsin system. The binding energy between IgG1 and the NP was calculated with the gmx energy tool.

### References for FIGS. 1, 21, 23-24

1. Duan et al. CD33 recruitment inhibits IgE-mediated anaphylaxis and desensitizes mast cells to allergen. J Clin Invest, 129(3). 1 Mar. 2019
2. Duan et al. Nanoparticles Displaying Allergen and Siglec-8 Ligands Suppress IgE-FcεRI-Mediated Anaphylaxis and Desensitize Mast Cells to Subsequent Antigen Challenge. J Immunol. ji1901212. 28 Apr. 2021.
3. Plum et al. Human Mast Cell Proteome Reveals Unique Lineage, Putative Functions, and Structural Basis for Cell Ablation. Immunity, 52(2). 18 Feb. 2020
4. Yu et al. Functional Inhibitory Siglec-6 Is Upregulated in Human Colorectal Cancer-Associated Mast Cells. Front. Immunol., 20 Sep. 2018
5. Du et al. Homopolymer self-assembly of poly(propylene sulfone) hydrogels via dynamic noncovalent sulfone—sulfone bonding. Nat. Commun, Volume 11, Article number: 4896. 29 Sep. 2020
6. Dispenza et al. Bruton's tyrosine kinase inhibition effectively protects against human IgE-mediated anaphylaxis. J Clin Invest. 130(9):4759-4770. 4 Aug. 2020
7. Caslin et al. The Use of Human and Mouse Mast Cell and Basophil Cultures to Assess Type 2 Inflammation. Methods Mol Biol. 1799:81-92. 29 Jun. 2018

### References for FIGS. 2, 77, 13, 20, 22

1 Walkey, C. D. & Chan, W. C. W. Understanding and controlling the interaction of nanomaterials with proteins in a physiological environment. Chemical Society Reviews 41, 2780-2799, doi:10.1039/C1CS15233E (2012).
2 Rabe, M., Verdes, D. & Seeger, S. Understanding protein adsorption phenomena at solid surfaces. Advances in Colloid and Interface Science 162, 87-106, doi:https://doi.org/10.1016/j.cis.2010.12.007 (2011).
3 Duan, S. et al. CD33 recruitment inhibits IgE-mediated anaphylaxis and desensitizes mast cells to allergen. The Journal of Clinical Investigation 129, doi:10.1172/JCI125456 (2021).
4 Duan, S. et al. Nanoparticles Displaying Allergen and Siglec-8 Ligands Suppress IgE-FcεRI—Mediated Anaphylaxis and Desensitize Mast Cells to Subsequent Antigen Challenge. The Journal of Immunology 206, 2290-2300, doi:10.4049/jimmunol.1901212 (2021).
5 Ramsey, A. V., Bischoff, A. J. & Francis, M. B. Enzyme Activated Gold Nanoparticles for Versatile Site-Selective Bioconjugation. Journal of the American Chemical Society 143, 7342-7350, doi:10.1021/jacs.0c11678 (2021).
6 Chen, J. et al. Tocilizumab—Conjugated Polymer Nanoparticles for NIR-II Photoacoustic-Imaging-Guided Therapy of Rheumatoid Arthritis. Advanced Materials 32, 2003399, doi:https://doi.org/10.1002/a dma.202003399 (2020).
7 Wang, X.-D., Rabe, K. S., Ahmed, I. & Niemeyer, C. M. Multifunctional Silica Nanoparticles for Covalent Immobilization of Highly Sensitive Proteins. Advanced Materials 27, 7945-7950, doi:https://doi.org/10.1002/adma.201503935 (2015).
8 Du, F. et al. Homopolymer self-assembly of poly(propylene sulfone) hydrogels via dynamic noncovalent sulfone—sulfone bonding. Nature Communications 11, 4896, doi:10.1038/s41467-020-18657-5 (2020).
9 Panganiban, B. et al. Random heteropolymers preserve protein function in foreign environments. Science 359, 1239-1243, doi:doi:10.1126/science.aao0335 (2018).
10 Galli, S. J., Gaudenzio, N. & Tsai, M. Mast Cells in Inflammation and Disease: Recent Progress and Ongoing Concerns. Annu Rev Immunol 38, 49-77, doi:10.1146/annurev-immunol-071719-094903 (2020).
11 Gotlib, J. et al. Proceedings from the Inaugural American Initiative in Mast Cell Diseases (AIM) Investigator Conference. J Allergy Clin Immunol 147, 2043-2052, doi:10.1016/j.jaci.2021.03.008 (2021).
12 Nel, A. E. et al. Understanding biophysicochemical interactions at the nano-bio interface. Nature Materials 8, 543-557, doi:10.1038/nmat2442 (2009).
13 Cao, Z.-T. et al. Protein Binding Affinity of Polymeric Nanoparticles as a Direct Indicator of Their Pharmacokinetics. ACS Nano 14, 3563-3575, doi:10.1021/acsnano.9b10015 (2020).
14 Estephan, Z. G., Jaber, J. A. & Schlenoff, J. B. Zwitterion-Stabilized Silica Nanoparticles: Toward Nonstick Nano. Langmuir 26, 16884-16889, doi:10.1021/1a103095d (2010).
15 Debayle, M. et al. Zwitterionic polymer ligands: an ideal surface coating to totally suppress protein-nanoparticle corona formation? Biomaterials 219, 119357, doi:https://doi.org/10.1016/j.biomaterials.2019.119357 (2019).
16 Tonigold, M. et al. Pre-adsorption of antibodies enables targeting of nanocarriers despite a biomolecular corona. Nature Nanotechnology 13, 862-869, doi:10.1038/s41565-018-0171-6 (2018).
17 Schöttler, S. et al. Protein adsorption is required for stealth effect of poly(ethylene glycol)- and poly(phosphoester)-coated nanocarriers. Nature Nanotechnology 11, 372-377, doi:10.1038/nnano.2015.330 (2016).
18 Kocbek, P., Obermajer, N., Cegnar, M., Kos, J. & Kristl, J. Targeting cancer cells using PLGA nanoparticles surface modified with monoclonal antibody. Journal of Controlled Release 120, 18-26, doi:https://doi.org/10.1016/j.jconrel.2007.03.012 (2007).
19 Sun, H. et al. Origin of Proteolytic Stability of Peptide-Brush Polymers as Globular Proteomimetics. ACS Central Science 7, 2063-2072, doi:10.1021/acscentsci.1c01149 (2021).
20 Qiao, B., Jimenez-Angeles, F., Nguyen, T. D. & Olvera de la Cruz, M. Water follows polar and nonpolar protein surface domains. Proceedings of the National Academy of Sciences 116, 19274-19281, doi:doi:10.1073/pnas.1910225116 (2019).
21 Kolkhir, P., Elieh-Ali-Komi, D., Metz, M., Siebenhaar, F. & Maurer, M. Understanding human mast cells: lesson from therapies for allergic and non-allergic diseases. Nature Reviews Immunology 22, 294-308, doi:10.1038/s41577-021-00622-y (2022).
22 Valent, P. et al. Drug-induced mast cell eradication: A novel approach to treat mast cell activation disorders? J Allergy Clin Immunol 149, 1866-1874, doi:10.1016/j.jaci.2022.04.003 (2022).
23 Balbino, B. et al. The anti-IgE mAb omalizumab induces adverse reactions by engaging Fcγ receptors. J Clin Invest 130, 1330-1335, doi:10.1172/jci129697 (2020).
24 Dispenza, M. C. et al. Bruton's tyrosine kinase inhibition effectively protects against human IgE-mediated anaphylaxis. The Journal of Clinical Investigation 130, 4759-4770, doi:10.1172/JCI138448 (2020).
25 Crocker, P. R., Paulson, J. C. & Varki, A. Siglecs and their roles in the immune system. Nat Rev Immunol 7, 255-266, doi:10.1038/nri2056 (2007).
26 Robida, P. A. et al. Functional and Phenotypic Characterization of Siglec-6 on Human Mast Cells. Cells 11, doi:10.3390/ce11s11071138 (2022).
27 Duan, S. et al. CD33 recruitment inhibits IgE-mediated anaphylaxis and desensitizes mast cells to allergen. J Clin Invest 129, 1387-1401, doi:10.1172/JCI125456 (2019).
28 Neuberger, M. S. et al. A hapten-specific chimaeric IgE antibody with human physiological effector function. Nature 314, 268-270, doi:10.1038/314268a0 (1985).
29 Bao, C. et al. A mast cell-thermoregulatory neuron circuit axis regulates hypothermia in anaphylaxis. Sci Immunol 8, eadc9417, doi:10.1126/sciimmunol.adc9417 (2023).
30 Bryce, P. J. et al. Humanized mouse model of mast cell-mediated passive cutaneous anaphylaxis and passive systemic anaphylaxis. J Allergy Clin Immunol 138, 769-779, doi:10.1016/j.jaci.2016.01.049 (2016).
31 Schanin, J. et al. Discovery of an agonistic Siglec-6 antibody that inhibits and reduces human mast cells. Communications Biology 5, 1226, doi:10.1038/s42003-022-04207-w (2022).
32 Thames, A. H. et al. A Cell-Free Protein Synthesis Platform to Produce a Clinically Relevant Allergen Panel. ACS Synthetic Biology 12, 2252-2261, doi:10.1021/acssynbio.3c00269 (2023).
33 Abraham, M. J. et al. GROMACS: High performance molecular simulations through multi-level parallelism from laptops to supercomputers. SoftwareX 1-2, 19-25, doi:https://doi.org/10.1016/j.softx.2015.06.001 (2015).
34 Huang, J. et al. CHARMM36m: an improved force field for folded and intrinsically disordered proteins. Nature Methods 14, 71-73, doi:10.1038/nmeth.4067 (2017).
35 Miyamoto, S. & Kollman, P. A. Settle: An analytical version of the SHAKE and RATTLE algorithm for rigid water models. Journal of Computational Chemistry 13, 952-962, doi:https://doi.org/10.1002/jcc.540130805 (1992).
36 Humphrey, W., Dalke, A. & Schulten, K. VMD: Visual molecular dynamics. Journal of Molecular Graphics 14, 33-38, doi:https://doi.org/10.1016/0263-7855(96)00018-5 (1996).
37 Caslin, H. L. et al. The Use of Human and Mouse Mast Cell and Basophil Cultures to Assess Type 2 Inflammation. Methods Mol Biol 1799, 81-92, doi:10.1007/978-1-4939-7896-0_8 (2018).

### References for FIGS. 3-6, 8-12, 14-19, 25-28

1 Du, F. et al. Homopolymer self-assembly of poly(propylene sulfone) hydrogels via dynamic noncovalent sulfone—sulfone bonding. Nature Communications 11, 4896, doi:10.1038/s41467-020-18657-5 (2020).
2 Martinez, L., Andrade, R., Birgin, E. G. & Martinez, J. M. PACKMOL: A package for building initial configurations for molecular dynamics simulations. Journal of Computational Chemistry 30, 2157-2164, doi:https://doi.org/10.1002/jcc.21224 (2009).
3 Essmann, U. et al. A smooth particle mesh Ewald method. The Journal of Chemical Physics 103, 8577-8593, doi:10.1063/1.470117 (1995).
4 Hess, B., Bekker, H., Berendsen, H. J. C. & Fraaije, J. G. E. M. LINCS: A linear constraint solver for molecular simulations. Journal of Computational Chemistry 18, 1463-1472, doi:https://doi.org/10.1002/(SICI)1096-987X(199709)18:12<1463::AID-JCC4>3 0.0. CO; 2-H (1997).
5 Hoover, W. G. Canonical dynamics: Equilibrium phase-space distributions. Physical Review A 31, 1695-1697, doi:10.1103/PhysRevA.31.1695 (1985).
6 Parrinello, M. & Rahman, A. Polymorphic transitions in single crystals: A new molecular dynamics method. Journal of Applied Physics 52, 7182-7190, doi:10.1063/1.328693 (1981).
7 Qiao, B., Demars, T., Olvera de la Cruz, M. & Ellis, R. J. How Hydrogen Bonds Affect the Growth of Reverse Micelles around Coordinating Metal Ions. The Journal of Physical Chemistry Letters 5, 1440-1444, doi:10.1021/jz500495p (2014).
8 Li, Y., Qiao, B. & Olvera de la Cruz, M. Protein Surface Printer for Exploring Protein Domains. Journal of Chemical Information and Modeling 60, 5255-5264, doi:10.1021/acs.jcim.0c00582 (2020).
9 Dispenza, M. C. et al. Bruton's tyrosine kinase inhibition effectively protects against human IgE-mediated anaphylaxis. The Journal of Clinical Investigation 130, 4759-4770, doi:10.1172/JCI138448 (2020).

### Sequences

| ID | Sequence |
|---|---|
| 1 | MQGAQEASASEMLPLLLPLLWAGALAQERRFQLEGPESLTVQEGLCVLVPCRL |
| | PTTLPASYYGYGYWFLEGADVPVATNDPDEEVQEETRGRFHLLWDPRRKNCSL |
| | SIRDARRRDNAAYFFRLKSKWMKYGYTSSKLSVRVMALTHRPNISIPGTLESGH |
| | PSNLTCSVPWVCEQGTPPIFSWMSAAPTSLGPRTTQSSVLTITPRPQDHSTNLTC |
| | QVTFPGAGVTMERTIQLNVSYAPQKVAISIFQGNSAAFKILQNTSSLPVLEGQAL |
| | RLLCDADGNPPAHLSWFQGFPALNATPISNTGVLELPQVGSAEEGDFTCRAQHP |
| | LGSLQISLSLFVHWKPEGRAGGVLGAVWGASITTLVFLCVCFIFRVKTRRKKAA |
| | QPVQNTDDVNPVMVSGSRGHQHQFQTGIVSDHPAEAGPISEDEQELHYAVLHF |
| | HKVQPQEPKVTDTEYSEIKIHK |
| 2 | MAPAMESPTLLCVALLFFAPDGVLAVPQKPKVSLNPPWNRIFKGENVTLTCNG |
| | NNFFEVSSTKWFHNGSLSEETNSSLNIVNAKFEDSGEYKCQHQQVNESEPVYLE |
| | VFSDWLLLQASAEVVMEGQPLFLRCHGWRNWDVYKVIYYKDGEALKYWYEN |
| | HNISITNATVEDSGTYYCTGKVWQLDYESEPLNITVIKAPREKYWLQFFIPLLVV |
| | ILFAVDTGLFISTQQQVTFLLKIKRTRKGFRLLNPHPKPNPKNN |

1. A method for adsorbing at least one protein onto the surface of a poly(propylene sulfone) (PPSU) homopolymer nanoparticle, the method comprising incubating the nanoparticle with the at least one protein in a buffer for between about 1 second and about 5 minutes at between about 20° C. and about 22° C.

2. The method of claim 1, wherein the concentration of the at least one protein is higher than the concentration of the nanoparticle.

3. The method of claim 1, wherein the concentration of the at least one protein is at least twice as high as the concentration of the nanoparticle.

4. The method of claim 1, further comprising washing the nanoparticle with the buffer between 1 and 3 times.

5. The method of claim 4, further comprising suspending the nanoparticle in the buffer.

6. The method of claim 1, wherein the buffer comprises phosphate buffered saline (PBS).

7. The method of claim 1, wherein the at least one protein is an antibody.

8. The method of claim 7, wherein the at least one antibody comprises two antibodies.

9. The method of claim 8, wherein the two antibodies comprise an anti-Siglec-6 antibody and an anti-FcεRI antibody.

10. The method of claim 9, wherein the anti-Siglec-6 antibody is antibody clone 767329, and wherein the anti-FcεRI antibody is antibody clone AER-37 (CRA1).

11. The method of claim 10, wherein the anti-Siglec-6 antibody is incubated at a density of between about 0.001 wt % and about 1000 wt % PPSU.

12. The method of claim 11, wherein the anti-FcεRI antibody is incubated at a density of between about 0.001 wt % and about 1000 wt % PPSU.

13. The method of claim 7, wherein after incubating the nanoparticle with the antibody, the method further comprises incubating the nanoparticle with bovine serum albumin (BSA).

14. The method of claim 1, wherein the PPSU homopolymer comprises PPSU₂₀.

15. A composition for reducing mast cell secretion, the composition comprising
a PPSU homopolymer nanoparticle coated with at least one of an anti-Siglec-6 antibody and an anti-FcεRI antibody; and
a pharmaceutically acceptable excipient, carrier or diluent.

16. The composition of claim 15, wherein the nanoparticle comprises the anti-Siglec-6 antibody and the anti-FcεRI antibody.

17. The composition of claim 15, wherein the nanoparticle comprises PPSU₂₀.

18. The composition of claim 15, wherein the anti-Siglec-6 antibody is antibody clone 767329, and wherein the anti-FcεRI antibody is antibody clone AER-37 (CRA1).

19. A method for treating a type I hypersensitivity reaction in a subject in need thereof, the method comprising administering a therapeutically effective amount of the composition of claim 15.

20. The method of claim 19, wherein the type I hypersensitivity reaction is anaphylaxis.
